(19) United States
(12) Patent Application Publication
(10) Pub. No.: US 2004/0133934 A1
(43) Pub. Date: Jul. 8, 2004

(54) Transgenic animals that produce human hemoglobin

(76) Inventors: Tim M. Townes, Birmingham, AL (US); Thomas Ryan, Birmingham, AL (US); Dominic Ciavatta, Birmingham, AL (US)

Correspondence Address: NEEDLE & ROSENBERG, P.C., SUITE 1000, 999 PEACHTREE STREET, ATLANTA, GA, 30309-3915, US

(21) Appl. No.: 10/659,675

(22) Filed: Sep. 10, 2003

Related U.S. Application Data
(63) Continuation of application No. 08/961,443, filed on Oct. 30, 1997, now abandoned.
Application No. 08/961,443 is a continuation-in-part of application No. 08/934,385, filed on Sep. 19, 1997, now abandoned.
Application No. 08/934,385 is a continuation-in-part of application No. 08/888,433, filed on Jul. 7, 1997, now abandoned.
Application No. 08/888,433 is a continuation of application No. 08/611,542, filed on Mar. 6, 1996, now abandoned.

(51) Int. Cl. A01K 67/027

(52) U.S. Cl. 800/18

(57) ABSTRACT

The invention features transgenic, non-human animals, such as mice, that produce human hemoglobin, for example, hemoglobin A, sickle hemoglobin, fetal hemoglobin, or hemoglobin Kansas Porto Alegre, but fail to produce hemoglobin endogenous to the animal. The invention also features methods for producing human hemoglobin in these animals, human hemoglobins produced by these methods, and methods of using these animals to identify therapeutic substances.

### CROSS REFERENCE TO RELATED APPLICATIONS

This application is a continuation-in-part of U.S. Ser. No. 08/934,385, filed on Sep. 19, 1997, which is a continuation-in-part of U.S. Ser. No. 08/888,433, filed on Jul. 7, 1997, which is a continuation of U.S. Ser. No. 08/611,542, filed on Mar. 6, 1996.

### BACKGROUND OF THE INVENTION

This invention relates to transgenic, non-human animals, such as mice, that produce human hemoglobin.

Hemoglobin, the oxygen carrying molecule of blood, is a tetramer of two α-globin subunits and two β-globin subunits. Each subunit contains a heme moiety, composed of a porphyrin ring and an iron atom, which binds oxygen. Specific alterations in hemoglobin gene sequences cause hematological disorders, such as thalassemias and sickle cell anemia. Thalassemias arise when the synthesis of an α- or β-globin subunit is absent or severely reduced. For example, in β-thalassemia, the ratio of β-globin subunits to α-globin subunits is less than 1. While the causes of thalassemias are heterogeneous, involving different mutations in the structural and regulatory sequences of the affected genes, sickle cell anemia is caused by a single nucleic acid substitution in the β-globin gene, which results in a Glu→Val substitution at position 6 of the β-globin subunit.

### SUMMARY OF THE INVENTION

In general, the invention provides transgenic, non-human mammals, such as mice, having erythrocytes that produce a human hemoglobin, but fail to produce a hemoglobin endogenous to the non-human mammal. A transgenic, non-human mammal of the invention can be used, e.g., for producing human hemoglobin, which can be used as a human blood substitute, or as an animal model system for a human hemoglobinopathy. Such animal model systems can be used for testing potential therapeutic drugs for efficacy in treating hemoglobinopathies, as well as for testing gene therapy methods.

The erythrocytes of a transgenic, non-human mammal of the invention produce human hemoglobin, but fail to produce hemoglobin (adult, non-adult, or both) endogenous to the transgenic, non-human mammal. As is described further below, the human hemoglobin made by the erythrocytes can be, e.g., human Hb A hemoglobin (Hb A; α₂/β₂), human sickle hemoglobin (Hb S; α₂/β^{S}₂), human fetal hemoglobin (Hb F; (α₂/γ₂), human anti-sickling hemoglobin (Kb AS; α₂/β^{AS}₂; e.g., Hb AS1-5, see below), or human P Kansas Porto Alegre hemoglobin (Hb KPA; α₂/β^{kpa}₂). In addition, combinations of these hemoglobins (e.g., Hb F+Hb S and Hb AS+Hb S) can be made in a transgenic, non-human mammal of the invention.

Nucleated precursors of the erythrocytes of a transgenic, non-human mammal of the invention can also have contained one or more human hemoglobin genes containing a mutation (or mutations) that is characteristic of a human thalassemic condition, optionally, in combination with a gene (or genes) encoding a wild type or mutant human hemoglobin, such as those described above. Numerous thalassemic mutations are well known in the art (see, e.g., Bunn et al., Hemoglobin: Molecular, Genetic, and Clinical Aspects (W. B. Saunders, Philadelphia, 1986)) and can be used in the invention.

Nucleated precursors of the erythrocytes of a transgenic, non-human mammal of the invention can also have contained a first chromosome including a human γ-globin gene and a human β-globin gene, and a second chromosome including a human ε-globin gene, a human γ-globin gene (e.g., two human γ-globin genes), a human δ-globin gene, and a human β-globin gene (e.g., a gene encoding a β^{s} hemoglobin chain).

Also included in the invention are transgenic, non-human mammals that have erythrocytes that produce one human hemoglobin chain (e.g., a human β-globin chain (e.g., the β-globin chain of Hb A, Hb S, an Hb AS (e.g., Hb AS1-5), or Hb KPA), a human γ-globin chain, or a human α-globin chain), but not one of the hemoglobin chains endogenous to the transgenic non-human mammal, as well as transgenic, non-human mammals that produce two human hemoglobin chains (e.g., any combination of a human β-globin chain (e.g., the β-globin chain of Hb A, Hb S, an Hb AS (e.g., Hb AS1-5), or Hb KPA), a human γ-globin chain, or a human α-globin chain), but not two of the hemoglobin chains endogenous to the transgenic mammal. These transgenic, non-human mammals can be used, for example, in crosses to produce mammals that express human hemoglobin, but not hemoglobin endogenous to the mammal. For example, the erythrocytes of a non-human, transgenic mammal of the invention can produce a human α-globin chain, a human β-globin chain, two human (C-globin chains, two human β-globin chains, one human α-globin chain and one human β-globin chain, or two human α-globin chains and two human β-globin chains. The erythrocytes can also fail to produce an α-globin chain endogenous to the transgenic, non-human mammal, a β-globin chain endogenous to the transgenic, non-human mammal, or any α-globin or β-globin chains endogenous to the transgenic, non-human mammal. In addition, the erythrocytes can produce two human α-globin chains and two human β-globin chains, but fail to produce any α-globin or β-globin chains endogenous to the non-human, transgenic mammal.

The transgenic, non-human mammals of the invention can be generated by crossing transgenic animals produced by gene knock out methods, which involve homologous recombination in embryonic stem cells, transgenic animals obtained by gene replacement using homologous recombination in embryonic stem cells, and transgenic animals obtained by microinjection of fertilized eggs. These methods are described further below.

The invention also provides methods for producing human hemoglobin. In these methods, human hemoglobin (e.g., human Hb A hemoglobin, human Hb F hemoglobin, human Hb AS hemoglobin, human Hb S hemoglobin, or human KPA hemoglobin) is expressed in the erythrocytes of a transgenic, non-human mammal of the invention, as is described above. Hemoglobin is purified from hemolysates of the transgenic, non-human mammal using standard methods.

Also included in the invention is human hemoglobin produced by a transgenic, non-human mammal described above. For example, the invention includes human Hb A hemoglobin, human Hb S hemoglobin, and human Hb KPA hemoglobin, produced by a transgenic, non-human mammal of the invention. The human hemoglobin can be stored in any appropriate buffer, such as phosphate-buffered saline.

The invention also includes methods of testing a substance for its efficacy in treating sickle cell anemia. In this method, a transgenic, non-human mammal, such as a mouse, that, preferably, expresses human sickle hemoglobin, but not hemoglobin endogenous to the non-human mammal, is exposed to the substance and a characteristic of sickle cell anemia, for example, the extent of red blood cell sickling, in the non-human mammal following substance exposure is monitored as a measure of the efficacy of the substance at treating sickle cell anemia. A decrease in red blood cell sickling, for example, indicates a substance that is useful for treating of sickle cell anemia. Methods for detecting red blood cell sickling in transgenic, non-human mammals of the invention are described further below. An example of a transgenic, non-human mammal that can be used in this method is one in which nucleated precursors of the mammal have contained a first chromosome including a human γ-globin gene and a human β-globin gene, and a second chromosome including a human ε-globin gene, a human γ-globin gene (e.g., two human γ-globin genes), a human 8-globin gene, and a human β-globin gene (e.g., a gene encoding a β^{s} hemoglobin chain).

In addition to identifying substances effective in treating sickle cell anemia by affecting red blood cell sickling, the methods of the invention can be used to identify substances effective in treating sickle cell anemia by other mechanisms. For example, substances that improve the clinical course of sickle cell anemia by, e.g., dilating capillaries can be identified using the methods of the invention.

The invention also includes methods for testing the efficacy of a substance in treating other hematological disorders, such as thalassemias. These methods employ a transgenic, non-human mammal (e.g., a mouse) that expresses a human hemoglobin gene containing a thalassemic mutation, but, preferably, not hemoglobin endogenous to the transgenic, non-human mammal. For example, for every two human α-globin chains, the transgenic, non-human mammal can produce one wild type β-globin chain and one β-globin chain containing a thalassemic mutation.

Cells (e.g., stem cells and cell lines derived from stem cells) derived from the transgenic, non-human mammals of the invention can also be used in screening methods for identifying substances useful for treating hematological disorders, such as sickle cell anemia and thalassemias. The invention includes these cells and cells lines, as well as substances identified using any of the above-described screening methods.

The term “human hemoglobin” is used herein to describe a polypeptide having an amino acid sequence that at least in part corresponds to the amino acid sequence of a naturally-occurring human hemoglobin molecule, whether mutated or wild type, and that has oxygen-carrying capacity.

The term “anti-sickling” is used herein to describe molecules that are capable of interfering with the aggregation of hemoglobin into the 14-stranded hemoglobin molecules characteristic of Hb S hemoglobin and resulting in sickle cell anemia. Preferably, the anti-sickling molecules of the invention have approximately the same anti-sickling properties as Hb F (i.e., α₂γ₂) hemoglobin (e.g., as measured by in vitro solubility assays, e.g., the assay of Benesch et al., J. Biol. Chem. 254:8169, 1979).

The terms “Hb S hemoglobin” and “β-sickling hemoglobin” are used herein to describe hemoglobin that aggregates into 14-stranded fibers at high intracellular concentrations and low partial pressure; such Hb S hemoglobin has an A to T transversion in the 6^{th} codon of the human β-globin gene. “Hb A” represents adult, human hemoglobin, which consists of two α-globin chains and two β-globin chains (α₂/γ₂); “Hb S” represents sickle hemoglobin, which consists of α₂/β^{s}₂; and “Hb F” represents fetal hemoglobin, which consists of two α-globin chains and two γ-globin chains (α₂/γ₂).

“Transgenic” is used herein to describe a non-human mammal that has a foreign (or partly foreign) gene incorporated into its genome, both in its germ cells and somatic cells. The foreign (or partly foreign) gene can have been introduced into the genome of the non-human mammal (or an ancestor of the non-human mammal) by homologous recombination (knock out or replacement) in an embryonic stem cell or by microinjection of a fertilized egg. Although transgenic mice are described throughout the application, other transgenic, non-human mammals, including, without limitation, rodents (e.g., hamsters, guinea pigs, rabbits, and rats), pigs, cattle, sheep, and goats are also included in the invention.

An advantage of the invention is that it facilitates production of human hemoglobins in the absence of hemoglobins from another species, such as the hemoglobin that the non-human transgenic mammal would naturally produce, but for the genetic modifications accomplished by the methods described below (i.e., endogenous hemoglobin). Thus, purification of the human hemoglobin from the non-human transgenic mammals of the invention is not complicated by fractionation of the human hemoglobin from the mammal's endogenous hemoglobin.

A related advantage of the invention is that hemoglobins produced in a transgenic, non-human mammal are not likely to be contaminated with human pathogens, such as hepatitis viruses or human retroviruses, such as HIV. Production of human hemoglobin offers the additional advantage of providing a human blood substitute that can be used to transfuse patients having any blood type, thus obviating the persistent problems created by limited availability of transfusable blood for rare or relatively unusual blood types.

An additional advantage of the invention is that it provides animal model systems that can be used, for example, to identify therapeutic substances. For example, a transgenic, non-human mammal that expresses human sickle hemoglobin, but not hemoglobin that is endogenous to the transgenic, non-human mammal, can be used in screening methods for identifying substances that can be used to treat sickle cell anemia. Similarly, a transgenic, non-human mammal that expresses a human hemoglobin gene containing a thalassemic mutation, but not hemoglobin that is endogenous to the transgenic, non-human mammal, can be used in screening methods to identify substances useful for treating thalassemias.

The transgenic, non-human mammals of the invention can be made using switching constructs (see below) that enable the mammals to exhibit hemoglobin expression patterns similar to those of humans. Generally, these constructs direct high levels of fetal hemoglobin expression in early development, and then direct a gradual switch to adult hemoglobin expression as development proceeds, leading to high levels of adult β-globin expression by the time the transgenic, non-human mammal is about one month old. Use of these constructs enables production of animal models of human hemoglobin expression.

The transgenic, non-human mammals of the invention that have erythrocytes, the precursors of which contained a chromosome containing a human β^{S}-globin switching construct, as well as a chromosome containing the full human β-globin locus (including a β^{S}-globin mutation; see above), are also useful in screening methods of the invention. These mammals allow identification of, for example, compounds useful in the activation of γ- and/or δ-globin expression, in the context of the intact human β-globin locus.

Other features and advantages of the invention will be apparent from the detailed description, the drawings, and the claims.

### BRIEF DESCRIPTION OF THE DRAWINGS

FIG. 1 is a schematic representation of the mating scheme for production of Hb A replacement mice.

FIG. 2 is a schematic representation of a strategy for making a β knock out/human β replacement mouse. LoxP sites (*) can be included on either side of the neo gene, in order to facilitate its post-antibiotic selection removal. The human β-globin gene can be a wild type human β-globin, or contain a mutation, such as the β-Kansas Porto Alegre mutation, a thalassemic mutation, or a sickle mutation.

FIG. 3 is a schematic representation of the mouse β-globin locus (line A) and replacement constructs (B-I), in which mouse sequences are replaced with the indicated human sequences.

FIG. 4 is a schematic representation of the mouse α-globin locus (line A) and replacement constructs (B-G), in which mouse sequences are replaced with the indicated human sequences.

FIG. 5 is a schematic representation of the mating scheme for production of transgenic Hb F→Hb A mice (doubly homozygous for mouse α-globin and β-globin deletions).

FIG. 6 is a photograph of transgenic mouse hemolysates fractionated by isoelectric focusing.

FIG. 7 is a schematic representation of a method for introducing a β^{S} mutation into the human β-globin locus in a Yeast Artificial Chromosome (YAC).

FIG. 8 is a schematic representation of a method for introducing a PS mutation into the human β-globin locus in a Bacterial Artificial Chromosome (BAC).

FIG. 9A is a schematic representation of the genomic organization of the human β- and α-globin loci.

FIG. 9B is a schematic representation of LCR γ-β^{S} and LCR α-globin constructs (see below) that were coinjected into fertilized eggs, as well as the structure of the wild type and knock out (Ciavatta et al., Proc. Natl. Acad. Sci. USA 92:9259-9263, 1995; Paszty et al., Nature Genet. 11:33-39, 1995) mouse ax- and β-globin loci (top and bottom, respectively). Mice carrying the transgenes shown in FIG. 9A were mated with mice harboring these targeted gene deletions to generate Hb S mα^{0/0}, mβ^{0/0} animals.

FIG. 9C is a photograph of a gel showing genotype analysis, by PCR amplification (see below), of tail DNA from a wild type mouse (lane 1), an Hb S transgenic mouse doubly heterozygous for the mouse α- and β-globin deletions (lane 2), and an Hb S transgenic mouse homozygous for both the mouse α- and β-globin deletions (lane 3). The orientation of the two lower gels (panels) is flipped vertically to be aligned with the schematic representations in FIG. 9B.

FIG. 10 is a photograph of nondenaturing, isoelectric focusing gel analysis of Hb S hemolysates. Hemolysates from four independent Hb S mα^{0/0}, mβ^{0/0} transgenic lines (Hb S2, Hb S3, Hb S4, and Hb S5) demonstrate that only human Hb S and human Hb F are synthesized in these adult animals. The outside lanes are mouse and human Hb SS controls.

FIGS. 11A-11D are a series of graphs showing reverse-phase HPLC profiles (see below) of adult hemolysates from a mouse control (FIG. 11A), Hb S3 double heterozygote (mα^{0/+}, mβ^{0/+}, FIG. 11B), and Hb S3 double homozygote (mα^{0/0}, mβ^{0/0}, FIG. 11C), which show the globin chains synthesized by these mice. FIG. 11D is a profile of a hemolysate from a human Hb SS individual. LCR α/LCR Aγ-β^{S} transgenic mice doubly homozygous for the murine α- and β-globin gene knock out alleles (Hb S3 mα^{0/0}, mβ^{0/0}, FIG. 1C) produce only human α-, γ-, and β^{S}-globin chains as adults. Quantitation of γ and β^{S} chains (see below) demonstrates that y polypeptides (Aγ only) are 6.7% of total γ+β^{S} chains in the Hb S3 mouse hemolysate, which is similar to 8.9% (Aγ+Gγ) calculated for the human Hb SS sample.

FIG. 12 is a photograph of microscopic analysis of peripheral blood smears of control and Hb S mice. The top panels illustrate Wright-Geimsa stains of control, Hb S2, and Hb S3 mice under normal oxygen tension. The bottom panels illustrate supravital staining of reticulocytes with New Methylene Blue and counter staining with Wrights stain.

FIG. 13 is a photograph of Hematoxylin/Eosin stained sections of control and Hb S mouse tissues. Low magnification (10×) and high magnification (250×) of spleen, liver, and kidney tissue sections are shown. Hb S spleens are characterized by massive expansion of erythroid precursors, pooling of sickled erythrocytes in the sinusoids, vascular occlusion, and thrombosis. Livers of Hb S animals exhibit extensive periportal and subcapsular focal necrosis, extramedullary hematopoiesis, and numerous sickled erythrocytes in the intrahepatic vasculature and sinusoids. In the kidneys of Hb S mice, vascular occlusion is most prominent in the corticomedullary junction, where dilated capillaries are engorged with sickled red blood cells. Tubular damage results in a decrease of urine concentrating ability.

FIGS. 14A and 14B are photographs of transmission electron microscopic analysis of erythrocytes in the splenic sinusoid of Hb S 3 mice. FIGS. 14A and 14B show sections cut parallel or perpendicular to the long axis of the Hb S fibers, respectively. Extensive human Hb S fiber formation is observed under normal oxygen tension. These intracellular Hb S fibers are indistinguishable from fibers observed in red blood cells of humans with sickle cell disease. The original magnification is 60,000×.

FIG. 15 is a series of graphs showing the pattern of hemoglobin switching in Hb A mice ranging from a 15.5 day fetus to a 9 week old adult.

FIG. 16 is a series of graphs showing the patterns of hemoglobin expression in Hb S3, Hb F, and Hb S3×Hb F mice.

### DETAILED DESCRIPTION

The invention provides transgenic, non-human mammals, such as mice, that produce human hemoglobin, but not hemoglobin that is endogenous to the non-human transgenic mammals. These mammals can be used for producing human blood substitutes (e.g., human hemoglobin Hb A (hα₂/hβ₂), human fetal hemoglobin (Hb F; hα₂/hγ₂), and human hemoglobin Kansas Porto Alegre (hα₂/hβ^{KPA}₂)), as well as for animal model systems of hemoglobinopathies, such as thalassemias and sickle cell anemia.

The mammals of the invention can be generated using combinations of genetic methods. For example, Section I, below, describes the use of homologous recombination to replace mouse α- and β-globin genes in mice with their human counterparts. Section II describes the use of knock out methods to delete mouse α- and β-globin genes from mice, and the use of microinjection into fertilized eggs to introduce genes encoding human (α- and β-globin genes into mice. Sections III, IV, and V describe methods that can be used, in combination with those described in Sections I and II, for generating mice expressing human sickle hemoglobin, human crosslinked hemoglobins, and human anti-sickling hemoglobins, respectively.

I. Human Hemoglobin Replacement Mice (hα/hα, hβ/hβ)

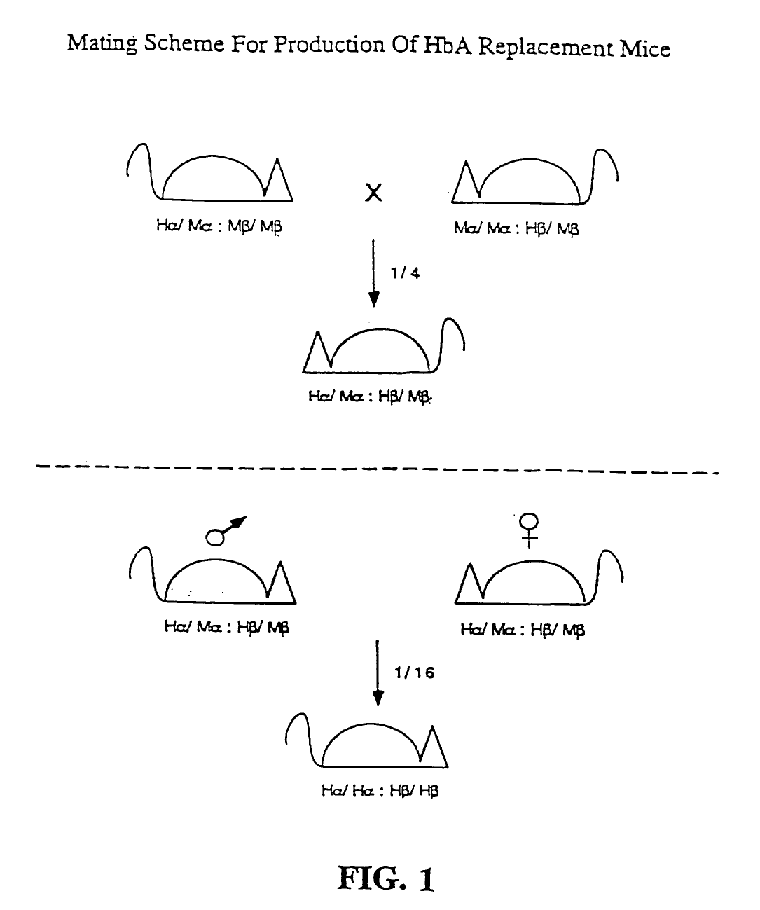

One method for making a mouse that produces human, but not murine, hemoglobin is illustrated in FIG. 1. Briefly, a mouse in which the endogenous α-globin genes (α1 and α2) on one chromosome have been knocked out and replaced with a human α-globin gene (or genes) by homologous recombination in embryonic stem cells (an “α-replacement heterozygote” or “hα/mα, mβ/mβ”) is crossed with a mouse in which the endogenous β-globin genes (β^{maj} and β^{min}) on one chromosome have been knocked out and replaced with a human β-globin gene (or genes) (a “β-replacement heterozygote” or “mα/mα, hβ/mβ”). Progeny of this cross, which are heterozygous for human α- and β-globin genes (a “double-replacement heterozygote” or “hα/mα, hβ/mβ”), are crossed with each other to generate a human hemoglobin replacement mouse (“double-replacement homozygote” or “hα/hα, hβ/hβ”). This method is described in further detail below.

Production of Single Replacement Heterozygotes (hα/mα, mβ/mβ and mα/mα, hβ/mβ)

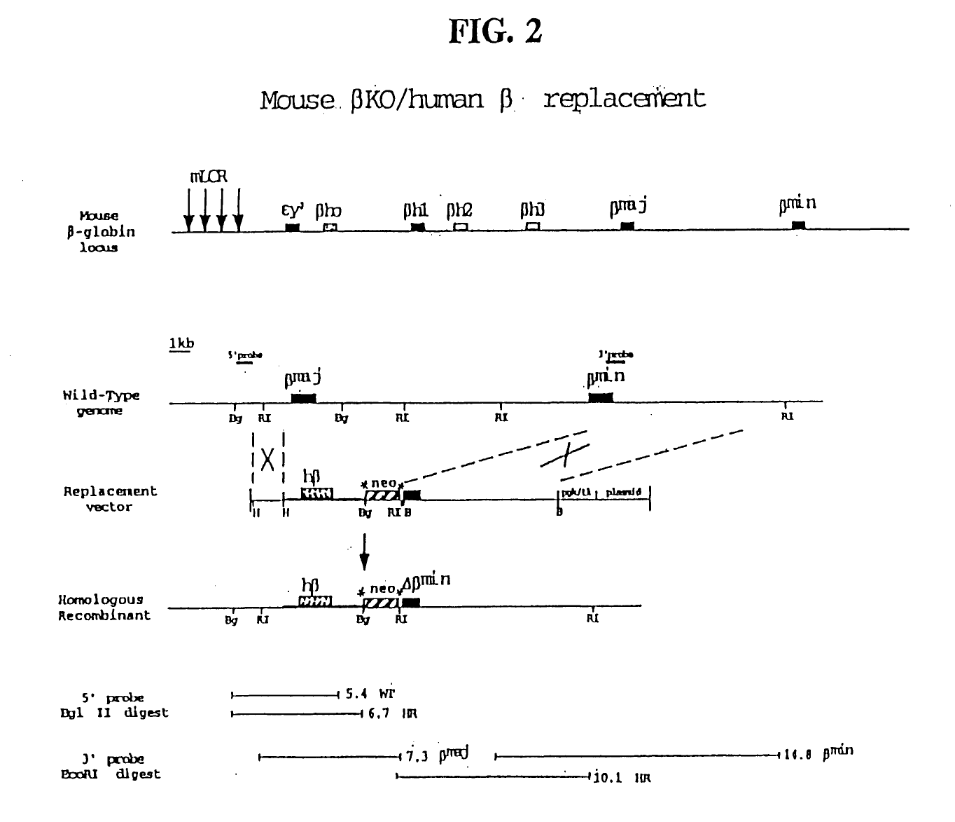

Gene replacement using homologous recombination in embryonic stem cells for production of replacement heterozygotes is carried out using standard genetic methods (see, e.g., Ausubel et al., eds. Current Protocols in Molecular Biology, Wiley & Sons, New York, 1989). In these methods, a replacement vector is constructed in which the replacement gene, together with a selectable marker, such as an antibiotic resistance gene, is flanked by sequences homologous to sequences that flank the target gene to be replaced. FIG. 2 illustrates the replacement vector that was used to replace murine β-globin genes with human β-globin genes. (See Ciavatta et al., Proc. Natl. Acad. Sci. USA 92:9259-9263, 1995, for details on a similar construct, used to delete, rather than replace, mouse β^{maj}- and β^{min}-globin genes in embryonic stem cells.) The replacement vector is transfected, e.g., by electroporation, into appropriate mouse embryonic stem cells, such as D3 (Ciavatta et al., supra), R1 (Nagy et al., Proc. Natl. Acad. Sci. USA 90:8424-8428, 1993), or RW4 (Genome Systems, St. Louis, Mo.) cells, and clones in which the replacement and selectable marker genes have replaced the target gene are selected by culturing the cells in selection medium. For example, when the neomycin resistance gene is used as a selectable marker, selection medium containing G418 can be used. Confirmation of homologous recombination can be carried out by Southern blot analysis of genomic DNA prepared from selected clones.

The selectable marker gene, e.g., an antibiotic resistance gene, such as a neomycin resistance gene, can be incorporated into the vector so that it can be removed after selection. For example, in constructing the replacement vector, the selectable marker gene can be flanked by loxP sites. Selected clones can then be transiently transfected with a gene encoding the cre enzyme, which catalyzes a loxP site-dependent recombination, so that the DNA sequences between the two loxP sites, i.e., the sequences corresponding to the selectable marker gene, are efficiently excised from the chromosome (Gu et al., Cell 73:1155-1164, 1993).

An additional method for ensuring removal of a selectable marker is the tag and exchange method (Stacey et al., Molecular and Cellular Biology 14(2):1009-1016, 1994). In this method, the targeted gene in the embryonic stem cells is replaced with the hypoxanthine phosphoribosyltransferase (HPRT) gene using standard gene replacement by homologous recombination in embryonic stem cells. Clones in which the HPRT gene has replaced the target gene are selected by culture in medium containing hypoxanthine aminopterin thymidine (HAT). In a subsequent round of homologous recombination, the HPRT gene is replaced with the replacement gene (e.g., a human α- or β-globin gene). Cells are then cultured in 6-thioguanine to select for clones that lack HPRT, and thus have undergone site-specific recombination to replace the HPRT gene with the replacement gene.

Using standard methods, embryonic stem cells that have been confirmed to have undergone the correct knock out and replacement are injected into blastocysts, such as C57BL/6 blastocysts, which are implanted into pseudo-pregnant female mice to generate chimeric mice. Male chimeric mice are bred with female mice, such as C57BL/6 female mice, to generate single replacement heterozygotes, which can be identified using, e.g., Southern blot analysis.

Production of a Double-Replacement Heterozygote (hα/mα, hβ/mβ)

In order to produce a double-replacement heterozygote (hα/mα, hβ/mβ), an α-replacement heterozygote (hα/mα, mβ/mβ) is crossed with a β-replacement heterozygote (mα/mα, hβ/mβ). Double-replacement heterozygotes produced by this cross can be identified using, e.g., Southern blot analysis.

Production of a Double-Replacement Homozygote (hα/hα, hβ/hβ)

A double-replacement homozygote (hα/hα, hβ/hβ) can be made by crossing two double-replacement heterozygotes (e.g., hα/mα, hβ/mβ). Double-replacement homozygotes, which express human, but not murine, α- and β-globin genes, can be identified using standard methods, such as Southern blot analysis.

Examples of Replacing Mouse β- and α-Globin Loci with Human Globin Genes

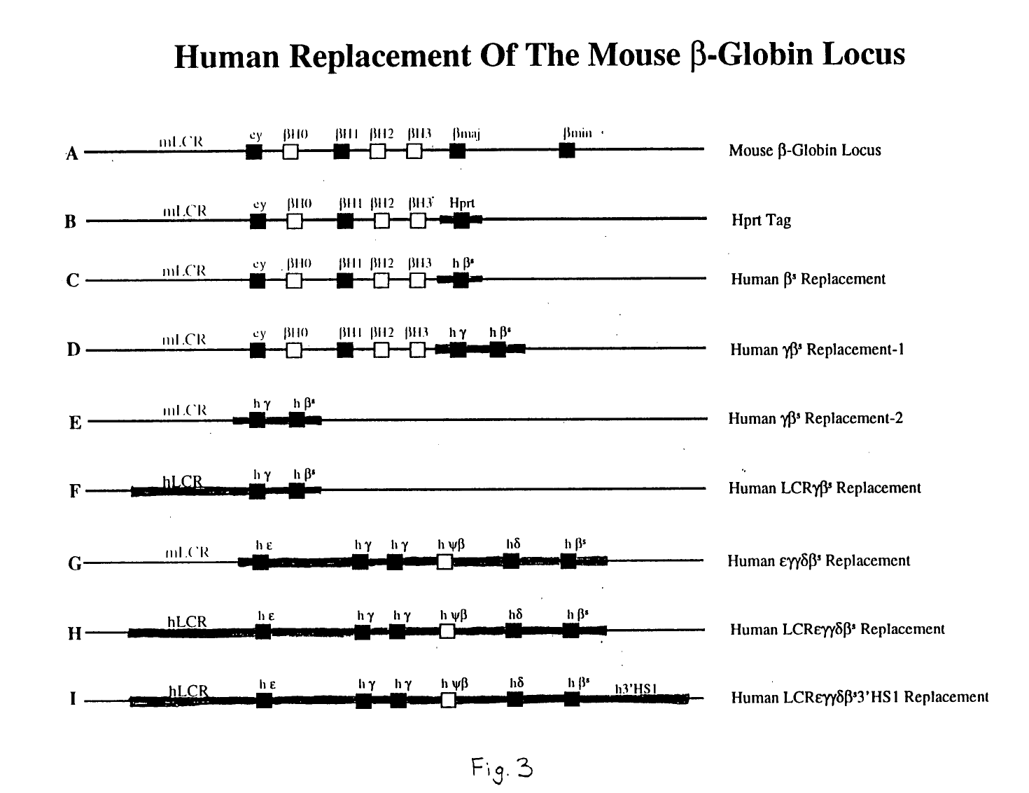

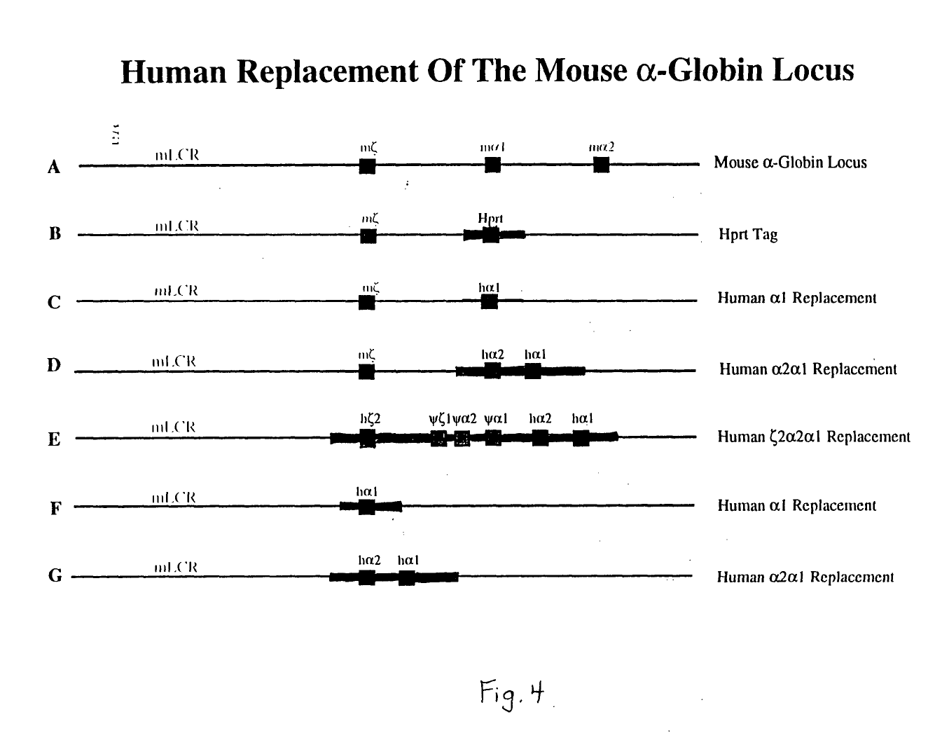

There are a variety of approaches that can be used to accomplish substitution of a human globin gene(s) for a globin gene(s) of an experimental animal (e.g., a mouse) using homologous recombination in embryonic stem (ES) cells. FIGS. 3 and 4 show examples of β- and α-globin mouse mutants that can be constructed using gene replacement strategies. A detailed description of the production and expectant phenotype of each mouse mutant shown in FIGS. 3 and 4 using a double replacement, or “in and out,” strategy (Stacey et al., Mol. Cell. Biol. 14(2):1009-1016, 1994) is described below. Other strategies, such as the “plug and socket” (Lewis et al., J. Clin. Invest. 97:3-5, 1996), “tag and exchange” (Askew et al., Mol. Cell. Biol. 13:4115-4124, 1993), “hit and run” (Wu et al., Proc. Natl. Acad. Sci. USA 91(7):2819-2823, 1994), and “positive-negative” selection (Mansour et al., Nature 336(6197):348-352, 1988), followed by cre mediated LoxP recombination (Sauer et al., Nucleic Acids Research 17(1): 147-161, 1989), can also be used to generate these mouse mutants.

“Tagging” the Mouse β- and α-Globin Loci

The top lines of FIGS. 3 and 4 (labeled as lines “A”) depict the endogenous mouse β- and α-globin loci, respectively. All murine sequences are drawn with thin lines; the functional genes at each locus are indicated by rectangles; the pseudogenes are indicated by “βHO,” “βH2,” or “βH3” on top of the rectangles. The second lines of FIGS. 3 and 4(B) represent loci that have been tagged with the selectable marker gene hypoxanthine phosphoribosyltransferase (Hprt). The hprt gene (mouse minigene) has been inserted into these loci via homologous recombination by cloning the murine sequences upstream and downstream from the adult globin genes on either side of the hprt gene. After electroporation into HM-1 ES cells (hprt-), clones are picked that have inserted the hprt DNA construct. These clones are identified by survival in Hypoxanthine/Aminopterin/Thymine (HAT) selection medium. Hprt-tagged loci that have recombined site-specifically into the globin loci by homologous recombination between endogenous sequences and the identical sequences in the Tag constructs can be identified by Southern analysis. As depicted in FIGS. 3 and 4, both of the adult globin genes are simultaneously deleted upon tagging the loci with hprt (compare lines A and B). Mice derived from these tagged cells express no murine globin chains in adult erythrocytes, similar to knock outs already described (Ciavatta et al., Proc. Natl. Acad. Sci. USA 92(20):9259-9263, 1995; Yang et al., Proc. Natl. Acad. Sci. USA 92(25):11608-11612, 1995; Paszty et al., Nat. Genetics 11(1):33-39, 1995).

“Exchange” of Murine Globin Genes With Human Globin Genes

These hprt-tagged ES cells can be used to generate the remaining replacement mutations (shown in FIGS. 3 and 4, lines C-I and C-G, respectively) by an “exchange” strategy. In each experiment, human sequences replace some or all of the endogenous murine globin genes and regulatory sequences. Human sequences are indicated by the thick lines in lines C-I of FIG. 3 and the thick lines in lines C-G of FIG. 4; the functional globin genes are indicated by the prefix “h”; the pseudogenes are indicated by the symbol “ψ.” Each “exchange” requires the construction of a DNA replacement construct that consists of the human globin sequences to be inserted into the mouse loci, flanked by the DNA surrounding the insertion site. Recombination between identical sequences in the replacement construct and in the ES cell genome results in the substitution of the deleted murine sequence by the human sequence. Cells in which these recombination events have occurred are selected in media containing 6-thioguanine (6-TG), which selectively kills ES cells that contain hprt. Homologous recombinants survive this selection because they have deleted the hprt gene.

Construction of the Human Replacement Mouse Mutants

Building the various replacement constructs shown in FIGS. 3 and 4 is relatively straightforward for the smaller plasmid and cosmid-based constructs. Regions of upstream and downstream mouse homology are simply spliced into the plasmid or cosmid on either side of the human sequence. This can be done for lines C, D, E, and F of FIG. 3 and lines C, D, F, and G of FIG. 4. The remaining constructs can be built using BAC (Bacterial Artificial Chromosome) clones containing the human β- and α-globin loci. Briefly, two successive rounds of targeted modification of the human BAC clone can be carried out (Yang et al., Nat. Biotech. 15:859-865, 1997). In the first round, one mouse homology region is inserted into a human DNA fragment located at one end of the desired region that is to be exchanged. This human DNA fragment, with its embedded murine homology region, is then cloned into the RecA shuttle vector. After co-integration and resolution with the human BAC clone, colonies are screened and mapped for those containing the mouse homology inserted into the human globin locus at the desired position. A second round of targeted modification of this newly mutated human BAC is performed identically to the first round, except that the second homology region is embedded into a human DNA fragment at the other end of the region that is to be exchanged. The end result is a human globin BAC clone with murine upstream and downstream homology regions inserted at the desired genomic endpoints. The entire; BAC clone is then electroporated into the hprt “tagged” ES cells, and homologous recombinants are identified after 6-TG selection. This strategy results in the replacement of the whole mouse β- and α-globin coding regions with the entire human β- and α-globin loci (FIG. 3, lines G and E, respectively). Additionally, the murine LCR can be substituted by the human LCR in FIG. 3, lines H and I. The largest “exchange” mutant can also include the human 3′ HSI site 20 kb downstream of the β-globin gene (FIG. 3, line I). This last replacement mutant (FIG. 3, line I) substitutes all of the known regulatory and gene coding sequences of the human β-globin locus for all of the known regulatory and gene coding sequences of the mouse β-globin locus.

Expression Pattern of the Human Globin Replacement Mice

The hprt “tagged” loci produces no adult murine globin genes. Initiation of human β^{s}-globin gene expression (FIG. 3, line C) occurs during fetal life beginning around day 12 of gestation, mimicking the endogenous murine embryonic to fetal/adult globin gene switch. Mutants with the human γβ^{s} and LCRγβ^{s} replacements (FIG. 3, lines D, E, and F) exhibit the same delayed fetal to adult globin gene switch seen in LCRγβ^{s} transgenic mice. The replacement mutants depicted in FIG. 3, lines G, H, and I, switch the expression of their globin genes similar to that seen for human YAC (Yeast Artificial Chromosome) or cosmid transgenic mice. All of the human x-globin replacement mutants (FIG. 4, lines C to G) express the adult human α1- and/or α2-globin gene early in embryonic life and remain on through adulthood.

II. Human Hemoglobin Producing Mice (Hb A mα⁰/mα⁰, mβ⁰/mβ⁰) Made by Crossing Knock Out Mice with Transgenic Mice Made by Microinjection

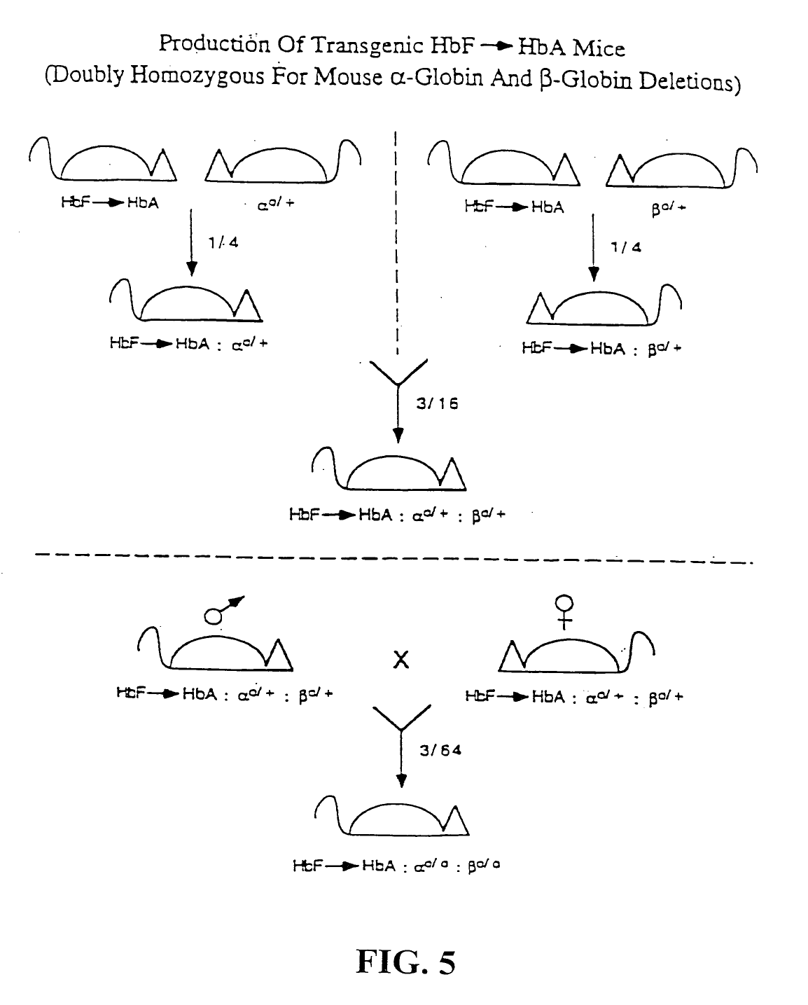

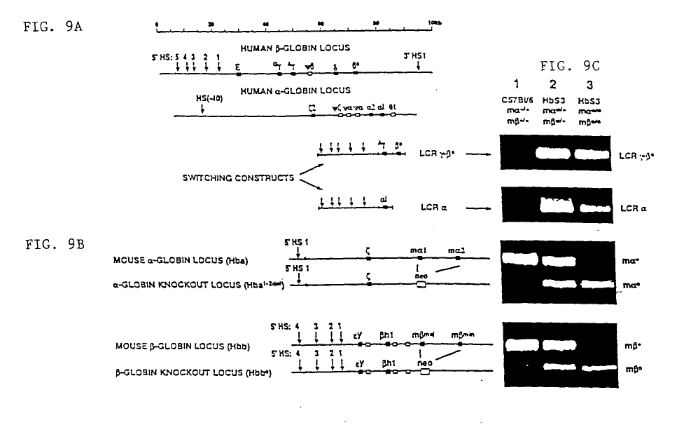

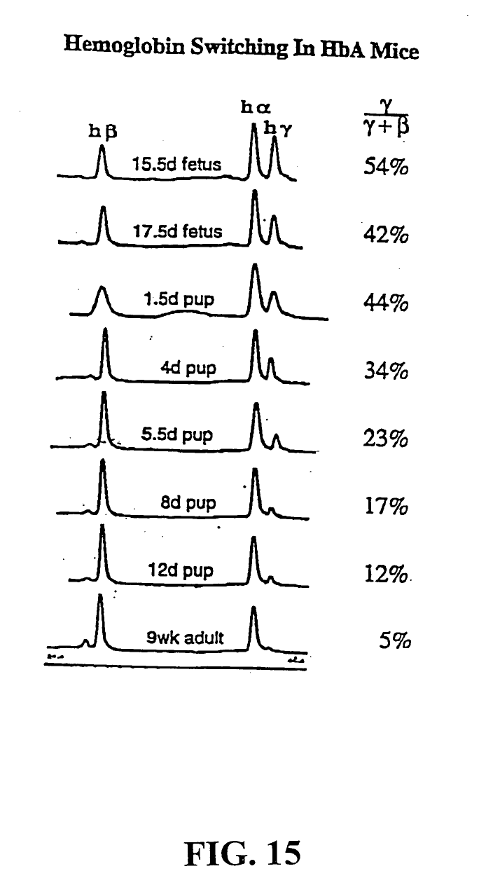

A second method for making a mouse that produces human, but not murine, hemoglobin is illustrated in FIG. 5. A mouse in which the endogenous α-globin genes on one chromosome have been knocked out, but not replaced, by homologous recombination in embryonic stem cells (an “α-knock out heterozygote” or “mα⁰/mα, mβ/mβ”; see, e.g., Paszty et al., Nature Genetics 11:33-39, 1995) is crossed with a mouse that was produced by microinjection of a fertilized egg with human α- and β-globin genes (Hb A, mα/mα, mβ/mβ; see FIG. 9A for an illustration of switching constructs that can be used to generate such a mouse; “β^{S}” in the β switching construct can be replaced with a wild type human β-globin gene). Similarly, a mouse in which the endogenous β-globin genes (β^{maj} and β^{min}) on one chromosome have been knocked out, but not replaced (a “β-knock out heterozygote” or “mα/mα, mβ⁰/mβ”; see, e.g., Ciavatta et al., supra; production of this mouse is described further below), is crossed with a mouse that was produced by microinjection of a fertilized egg with human α- and β-globin genes (Hb A, mα/mα, mβ/mβ; see FIG. 9A and above). As is mentioned above, the construct used for microinjection can be a so-called “switching construct,” which directs high levels of human fetal hemoglobin expression during early development and gradually “switches” to direct high levels of adult β-globin gene expression by approximately one month of age. Patterns of hemoglobin switching in Hb A mice are illustrated in FIG. 15. The switching constructs are described further below and are illustrated, in reference to Hb S, in FIG. 9A. Progeny from this cross, which are heterozygous for murine α- and β-globin genes (“double knock out heterozygotes” or “α⁰/mα, β⁰/mβ”), are crossed with each other to generate a mouse that produces human hemoglobin, but not murine hemoglobin (Hb A α⁰/α⁰, β⁰/β⁰).

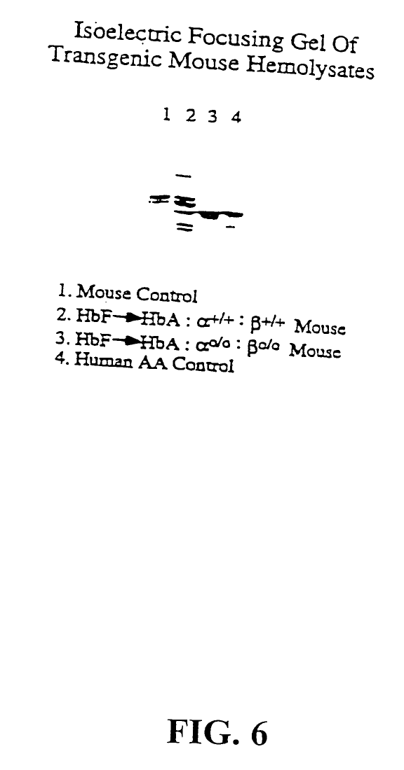

Using this method, we have generated such a mouse. Hemolysates from the mouse were fractionated by isoelectric focusing. As shown in FIG. 6, the mouse generated by these experiments produces human hemoglobin, but not murine hemoglobin.

Production of mice in which the β^{maj} and β^{min} globin genes have been knocked out is described as follows. These mice can be used in the crosses described above in order to generate mice that produce human, but not mouse, hemoglobin (e.g., Hb A or hemoglobin Kansas Porto Alegre). These mice can also be used as animal models for thalassemias, such as β⁰-thalassemia, which is an inherited disorder characterized by the absence of β-globin polypeptides derived from the affected allele. The molecular basis for this deficiency is a mutation of the adult β-globin structural gene or the cis regulatory elements that control β-globin expression.

We have produced a mouse in which both adult β-like globin genes, β^{maj} and β^{min}, are deleted. Heterozygous animals derived from the targeted cells are severely anemic, with dramatically reduced hemoglobin levels, abnormal red cell morphology, splenomegaly, and markedly increased reticulocyte counts. Homozygotes die in utero; however, heterozygous mice are fertile and transmit the deleted allele to progeny. The anemic phenotype is completely rescued in progeny derived from mating β⁰-thalassemic animals with transgenic mice expressing high levels of human hemoglobin A. As mentioned above, the β⁰-thalassemic mice can be used to test therapies, such as genetic therapies, for β⁰-thalassemia. They can also be bred with transgenic mice expressing high levels of human hemoglobin Hb S to produce a mouse model of sickle cell disease (see below), or with mice expressing hemoglobin Kansas Porto Alegre (see below) or human hemoglobin A (see above), in order to generate mice that can be used to produce blood substitutes. Methods for carrying out these experiments are described below. Additional details are described by Ciavatta et al. (supra).

Targeting Vector Construction

Homologous sequences flanking β^{maj} and β^{min}-globin genes were isolated from a 129 Sv/Ev strain mouse genomic library by using a 2.5 kb PstI probe containing β^{maj} globin gene sequences. The targeting vector was constructed by inserting a 1.7 kb HindIII fragment and a 7.0 kb BamHI fragment into the HindIII and BamHI sites of the plasmid pNTK (Mortensen et al., Mol. Cell Biol. 12:2391-2395; Ausubel et al., supra).

ES Cell Transfection and Characterization of a Homologous Recombinant

The targeting vector was linearized with SalI and introduced into the D3 line of ES cells, as described (Doetschman et al., J. Embryol. Exp. Morphol. 87:27-45, 1985). Briefly, 2×10⁷ cells in 1 ml of Dulbecco's modified Eagle's medium with 15% (vol/vol) fetal calf serum (HyClone) were electroporated with 25 μg of linearized vector DNA in a 0.4 cm cuvette at 400 V and 250 μF with a Bio-Rad Gene Pulsar. Twenty four hours after electroporation, cells were selected (Mansour et al., Nature 366:348-352, 1988) in G418 (300 μg/ml) and 2.5 μM gancyclovir (Syntex, Palo Alto, Calif.) for 2 weeks. Forty colonies were picked and expanded, and DNA was isolated for Southern blot analysis. The 5′ probe was a 1.45 kb Sau3A-HindIII fragment and the 3′ probe was a 1.12 kb BamHI-PstI fragment.

Characterization of Chimeras and Agouti Offspring

The probe used for Southern blot analysis of chimeras and agouti offspring was a 1.03 kb HindIII fragment from positions −340 to +690 of β^{maj}. This probe cross-hybridizes with β^{min}, β^{s}, and β¹ sequences from the BamHI site in the second exon to the end of this exon. Cellulose acetate gel electrophoresis (Behringer et al., Science 455:971-973, 1989) and primer-extension analysis were performed as described (Behringer et al., Genes Dev. 4:380-389, 1990). Primer-extension reaction mixtures contained 4 kg of RNA from 10 day yolk sac or 50 ng of RNA from adult blood. Bands were quantitated on a Molecular Dynamics PhosphorImager using IMAGEQUANT software.

Production of mice in which both α-globin genes have been knocked out is described as follows. Like the β-globin knock out mice, these mice can be used in the crosses described above in order to generate mice that produce human, but not mouse, hemoglobin (e.g., Hb A or hemoglobin Kansas Porto Alegre). Using gene targeting in mice, Paszty et al., (supra) deleted a 16 kb region encompassing both (X-globin genes. A mouse generated using the method of Paszty et al. can be used in carrying out crosses described above. The method and constructs described by Paszty et al. can also be used to produce additional α-knock out mice, as well as to replace mouse α-globin genes with human α-globin genes (Lauer et al., Cell 20:119-130, 1980) using the methods described above.

Production of Mice Containing a Human Switching Construct and the Full Human β-Globin Locus

The precise regulatory sequences that control human γ- to β-globin gene switching are unknown. The correct switching of the LCR γ-β transgene described above suggests that most if not all of the necessary sequences are located in the DNA fragments included in the LCR γ-β construct (FIG. 3). However, sequences located outside of these fragments may contribute to temporal-specific expression. To address this issue, the sickle mice described above can be bred with transgenic mice containing YAC or BAC clones encompassing the entire human β^{S}-globin locus (LCR ε-^{G}γ-^{A}γ-ψβ-δ-β^{S}). Mice containing LCR α/LCR ^{A}γβ^{S} and LCR ε-^{G}γ-^{A}γ-ψβ-δ-β^{S} transgenes and knockout mutations in the mouse α- and β-globin genes (mα^{0/0} and mβ^{0/0}) survive the perinatal period, because of γ expression from the LCR ^{A}γ-β^{S} tansgene. In adult mice, the human γ-globin genes in both transgene constructs are down-regulated, and the mice develop sickle cell disease. Strategies designed to reactivate the γ gene by drug or gene therapy can be tested using these mice. If γ in the LCR ε-^{g}γ-^{A}γ-ψβ-δ-β^{S} transgene is reactivated with different kinetics than y in the LCR ^{A}γ-β^{S} transgene, the mice can be used for determining optimal drug doses and to test gene therapies to reactivate the γ-globin gene in the context of the entire human globin locus. The mice can also be used in testing drug or gene therapies designed to enhance expression of the human δ-globin gene, which encodes a potent anti-sickling polypeptide.

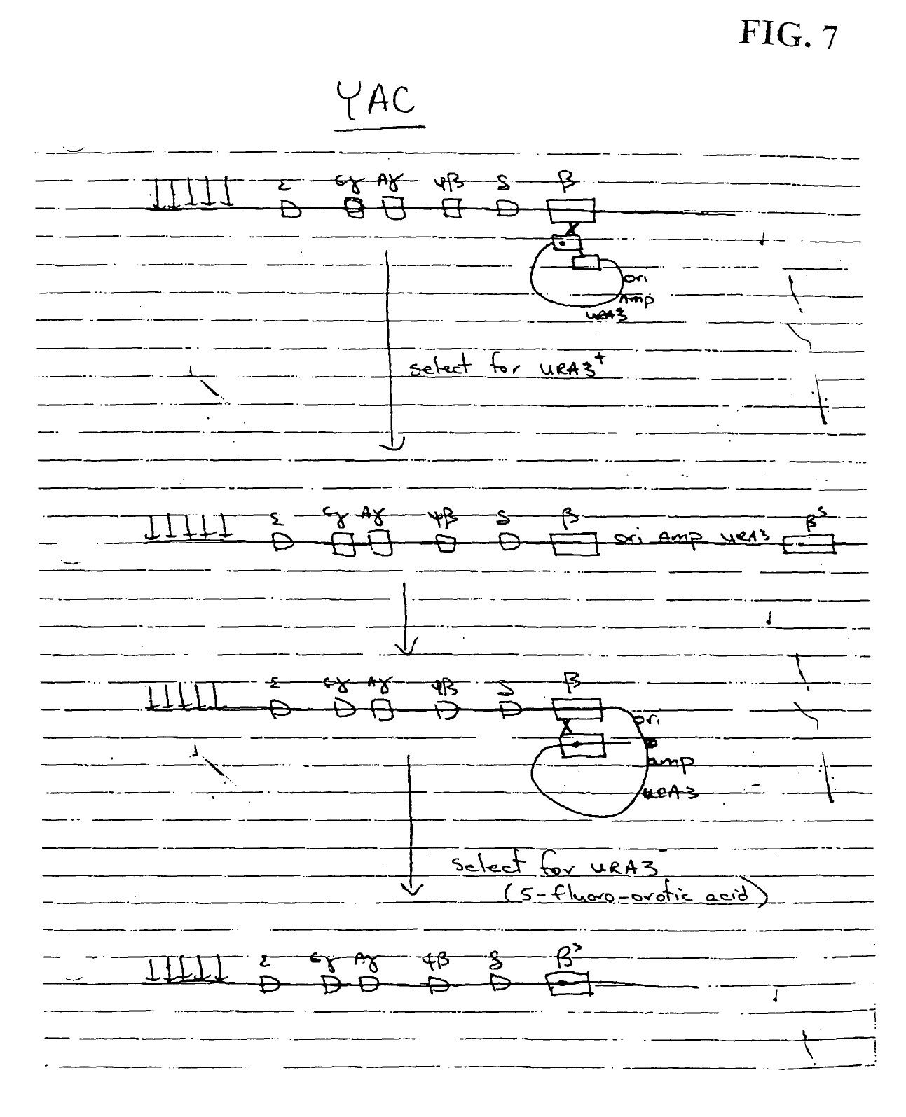

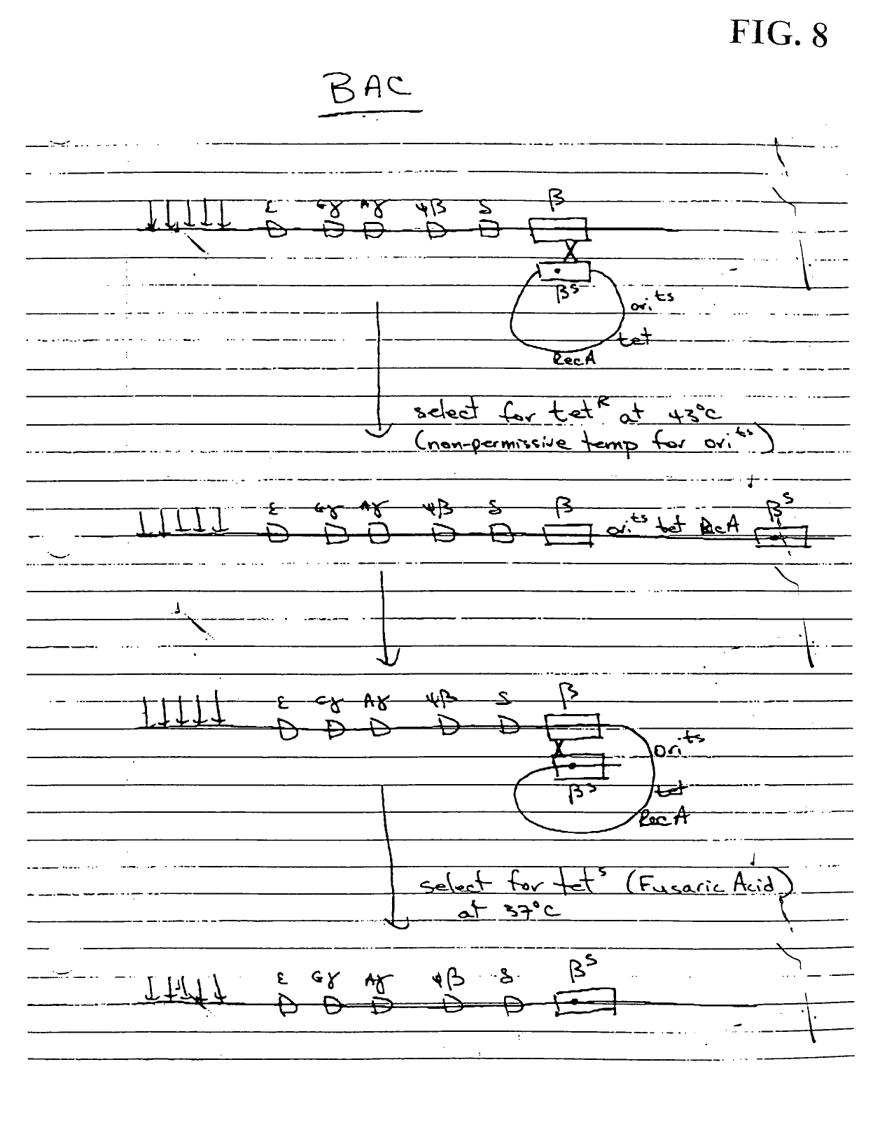

Transgenic mice that contain YAC or BAC transgenes are prepared using standard methods, for example, as described by Pederson et al. (Trends in Genetics 13:61-66, 1977, and references cited therein) and Yang et al. (Nature Biotechnology 15:859-865, 1997), respectively. YAC and BAC clones containing the entire human β-globin locus can be obtained from Genome Systems, Inc. (St. Louis), and the sickle mutation can be introduced into the β-globin gene by the standard procedures described in the papers cited above and illustrated in FIGS. 7 and 8. A point mutation in the YAC or BAC ^{A}γ gene can also be made by the methods described above so that ^{A}γ polypeptides produced from the LCR ε-^{g}γ-^{A}γ-ψβ-δ-β^{S} transgene can be distinguished from ^{A}γ polypeptides produced from the LCR ^{A}γ-β^{S} transgene.

Combination of Gene Replacement, Knockout, and Transgenic, Non-Human Mammals

Transgenic, non-human mammals synthesizing 100% human hemoglobin in their erythrocytes can be generated by a combination of gene replacement, gene knockout, and human transgene expression, as described above. These methods can be used to generate any native or recombinant hemoglobin (for example, see the hemoglobin types listed above) exclusively in the erythrocytes of a non-human mammal. Gene replacement at the endogenous globin loci, as described above, can also be used to generate animals that synthesize human hemoglobins exclusively. Furthermore, by mating together gene replacement mutants with knockout-transgenic mutant mice, the relative human globin chain ratios (α/β ratio), globin chain type (HbA, HbS, HbF, AS3, etc.), and timing of globin gene switching can all be manipulated to produce a desired phenotype.

III. P-Sickling Hemoglobins

As mentioned above, the invention provides a mouse model of human sickle cell disease. Transgenic mice were produced with constructs designed to direct Hb F (fetal hemoglobin) to Hb S (adult sickle hemoglobin) switching similar to humans. These transgenic animals were mated with mice containing knock out mutations of the adult mouse o-globin genes and the adult mouse β-globin genes to produce animals that synthesize only human hemoglobin in adult red blood cells. Similar to many human patients with sickle cell disease, the mice develop a severe hemolytic anemia and suffer extensive organ pathology. Numerous sickled erythrocytes are observed in peripheral blood, and sickled red blood cells occlude small capillaries and sinuses of the spleen, liver, and kidneys. Glomerular and tubular damage in the kidney result in loss of urine concentrating ability. Although chronically anemic, most animals survive for 2 to 9 months and are fertile. Drug and genetic therapies can be tested in this mouse model of sickle cell anemia. These experiments are described further, as follows.

The molecular basis for sickle cell disease is an A to T transversion in the 6^{th} codon of the human β-globin gene. This simple transversion changes a polar glutamic acid residue to a non-polar valine (Ingram, Nature 178:792-794, 1956; ibid, 180:326-328, 1957) in the β-globin polypeptide and the mutation drastically decreases the solubility of deoxygenated sickle hemoglobin. When the intracellular concentration of Hb S is high and the partial pressure of oxygen is low in the capillary beds, the non-polar valine, which is on the surface of the hemoglobin molecule, interacts with 2 other non-polar residues on the surface of a second hemoglobin molecule and initiates aggregation (Padlan et al., J. Biol. Chem. 260:8280-8291, 1985; Wishner et al., J. Mol. Biol. 98:179-194, 1975). Once approximately 10 hemoglobin molecules interact, long polymers rapidly accumulate and complex 14 stranded fibers are formed (Wood et al., Nature 313:320-326, 1985; Crepeau et al., Nature 274:616-617, 1978; Dykes et al., J. Mol. Biol. 130:451-472, 1979; Hofrichter et al., Proc. Natl. Acad. Sci. USA 71:4864-4868, 1974; Eaton et al., Blood 70:1245-1266, 1987). The formation of these fibers reduces the flexibility of red blood cells and leads to the occlusion of small capillaries. Intracellular fiber formation also results in erythrocyte membrane damage and increased red cell lysis (Noguchi et al., Blood 58:1057-1068, 1981; Brittenham et al., Blood 65:183-189, 1985). The ensuing disease is characterized by a chronic hemolytic anemia with episodes of severe pain and tissue damage that often result in kidney failure, liver disease, stroke, infection due to splenic infarction, and other complications.

Sickle cell disease is a relatively benign disorder in the first few months of life because of the potent antisickling properties of human fetal hemoglobin (Hb F) (Stamatoyannopoulos et al., The Molecular Basis of Blood Diseases (W. B. Saunders, Philadelphia, 1994); Bunn et al., Hemoglobin: Molecular, Genetic, and Clinical Aspects (W. B. Saunders, Philadelphia, 1986)). Hb F, which comprises 70-90% of total hemoglobin at birth, is gradually replaced by Hb S during the first few months of life. Rising Hb S levels result in the onset of disease between 3 and 6 months of age. A comprehensive mouse model of sickle cell disease would mimic the temporal switch of hemoglobins in man; however, none of the previous transgenic mouse models contained the human fetal (γ) globin gene. Large constructs containing human fetal and adult globin genes in their genomic context have been tested in mice. Animals containing cosmid and YAC transgenes (Behringer et al., Genes & Dev. 4:380-389, 1990; Enver et al., Nature 344:309-313, 1990; Gaensler et al., Proc. Natl. Acad. Sci. USA 90:11381-11385, 1993; Peterson et al., Proc. Natl. Acad. Sci. USA 90:7593-7, 1993; Strouboulis et al., Genes & Dev. 6:1857-1864, 1992) complete the switch from human γ- to β-globin gene expression by 15 days of gestation (6 days before birth). We reasoned that a switch to human Hb S at this early stage of development might decrease viability. To circumvent this potential perinatal lethality, DNA constructs designed to delay hemoglobin switching to approximate the fetal to adult globin gene switch in man were produced and tested in mice (Roberts et al., Blood 89:713-723, 1997).

The switching construct used to produce transgenic mice is illustrated in FIG. 9A. A 22 kb DNA fragment encompassing the human β-globin Locus Control Region (LCR) was linked to a 9.7 kb DNA fragment containing the Aγ-globin and β^{S}-globin genes. The LCR fragment was also linked to a 3.8 kb fragment containing the 0:1-globin gene. (In addition to the switching construct illustrated in FIG. 9A, other switching constructs, containing more or less 5′ flanking sequence, can be used in the invention and can readily be identified by one skilled in the art. For example, a construct containing a 5′ flanking sequence up to −1348, -383, -202, -160, or -130 can be used.) Transgenic mice produced by coinjecting these constructs were subsequently bred to the mouse at and β-globin knock out lines (FIG. 9B). Mice heterozygous for the transgenes and heterozygous for both the knock out loci were interbred to produce transgenic animals that were homozygous for the knock out alleles. PCR analysis (FIG. 9C) and Southern blot hybridizations confirmed the genotype of these animals. The mice express high levels of human fetal hemoglobin (Kb F) in the yolk sac blood islands early in development (8.5 d) and initiate a switch to human sickle hemoglobin (Hb S) when the site of hematopoiesis shifts to the fetal liver. At birth these animals continue to synthesize high levels of Hb F (40-50% of total human hemoglobin) and complete the switch to Hb S at approximately one month of age.

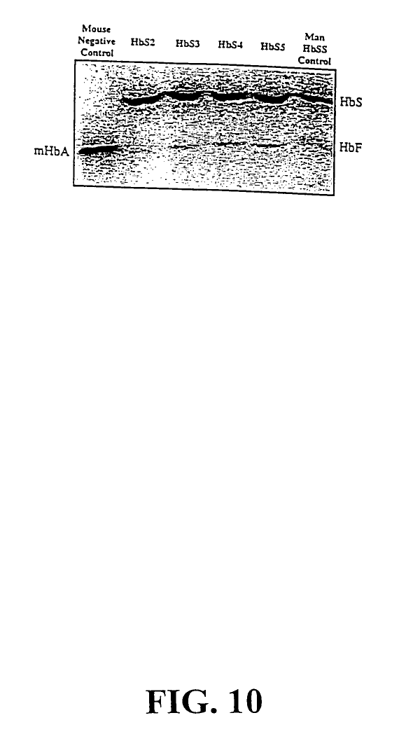

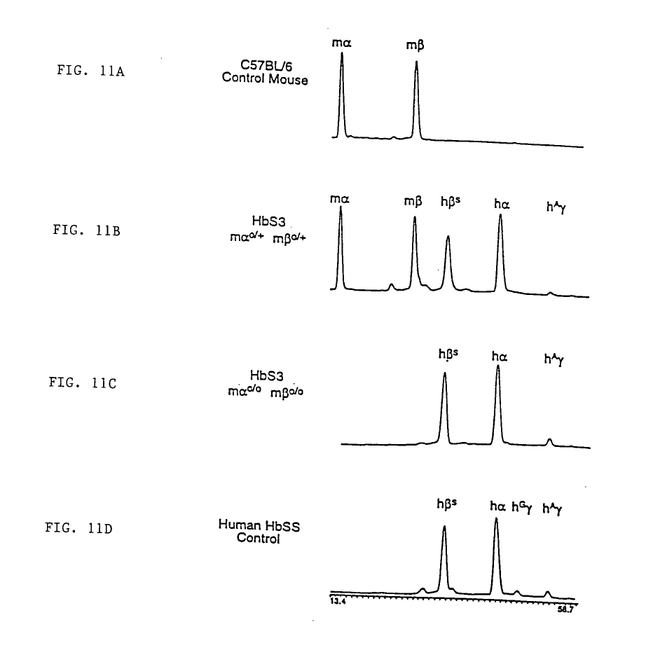

Adult Hb S transgenic animals that are homozygous for both of the knock out alleles (Hb S mα^{0/0}, mβ^{0/0}) synthesize no murine hemoglobin. An isoelectric focusing gel of hemolysates from 4 of the 6 lines that we produced is illustrated in FIG. 10. Only 2 bands are observed in each of the samples, and these proteins comigrate with human Hb S and human Kb F. FIGS. 11A-11D compare the HPLC profiles of hemolysates from one sickle cell mouse line (Kb S3) (FIGS. 11B and 11C) to hemolysates of a mouse control (FIG. 11A) and a human Kb SS control (FIG. 11D). Only human α-, ^{A}γ-, and β^{S}-globin chains are observed in LCR a/LCR ^{A}γ-β^{S} transgenic mice that are doubly homozygous for the murine α- and β-globin gene knock out alleles (Hb S3 mα^{0/0}, mβ^{0/0}) (FIG. 11C). Quantitation of γ and β^{S} chains demonstrate that γ polypeptides (^{A}γ only) are 6.7% of total γ+β^{S} chains and this value is similar to the human control, which is 8.9% (^{A}γ+^{G}γ). Gamma polypeptide levels were determined for all 6 Hb S lines and these values are listed in Table 1. The mean values for each line range from 3.2 to 8.0%. Average Hb F levels in homozygous sickle cell (Hb SS) patients is 5.8%, with a range between 0.4 and 18.8% (Serieant, Brit. J. Hemat. 19:635-641, 1970). The similarity of γ chain values in adult Hb S mice and human Hb SS patients significantly strengthens the model.

TABLE 1
Hematological parameters, human γ chain levels, spleen mass, and urine osmolality of Hb S mice. The
red blood cell counts, hemoglobin levels, and packed cell volumes were measured for control animals and five Hb S
lines. Reticulocyte counts were determined manually from smears of peripheral blood after vital staining with New
Methylene Blue. Human γ chain levels were calculated from the HPLC profiles of hemolysates and expressed as
the percentage of total γ plus β^{s} chains. Spleen mass is expressed as the percentage of the animals total body
weight. The individual values (n = 1 or 2) or the mean value ±SD (n ≧ 3) for each measurement is listed for each
group of animals.

| | | | | | | | Urine |
|---|---|---|---|---|---|---|---|
| | RBC | | | Reticulocytes | | Spleen (% | Osmolality |
| Line | (×10⁶/μL) | Hb (g/dL) | PCV (%) | (%) | γ/γ + β^{s} (%) | body wgt.) | (mOsm) |
| Control | 8.5 ± 0.9 (n = 7) | 15.0 ± 0.8 (n = 7) | 46 ± 2.6 (n = 7) | 3.9 ± 1.1 (n = 7) | NA* | 0.5 ± 0.2 (n = 7) | 1541 ± 360 |
| | | | | | | | (n = 6) |
| Hb S1 | 3.6 ± 0.7 (n = 3) | 4.1 ± 0.8 (n = 3) | 24 ± 3.8 (n = 3) | 56 ± 16 (n = 8) | 3.2 ± 0.6 (n = 11) | 7.1 ± 1.2 (n = 5) | ND |
| Hb S2 | 2.5 ± 0.8 (n = 3) | 4.5 ± 0.9 (n = 3) | 18 ± 4.5 (n = 3) | 68 ± 5 (n = 5) | 3.9 ± 1.9 (n = 9) | 7.6 ± 1.2 (n = 6) | ND |
| Hb S3 | 3.3 ± 1.0 (n = 5) | 6.1 ± 1.2 (n = 5) | 22 ± 4.7 (n = 5) | 57 ± 16 (n = 5) | 7.7 ± 2.2 (n = 13) | 5.9 ± 2.6 (n = 6) | 807 ± 285 |
| | | | | | | | (n = 7) |
| Hb S4 | 1.8 and 4.4 | 3.2 and 7.9 | 13 and 30 | 77 and 36 | 6.7 ± 0.6 (n = 3) | 5.6 and 4.0 | ND |
| Hb S5 | 2.4 and 3.8 | 5.2 and 7.5 | 20 and 29 | 82 and 44 | 6.0 ± 1.7 (n = 9) | 8.6 and 5.2 | ND |
| Hb S6 | ND^{#} | ND | ND | 60 | 4.5 | ND | ND |

*NA, not applicable;
^{#} ND, not determined

The hematological data listed in Table 1 demonstrate that knock out/transgenic animals develop a severe hemolytic anemia. All Hb S animals have a marked reduction in red blood cell (RBC) counts, hemoglobin (Hb) levels, and packed cell volumes (PCVs), and have significantly increased reticulocyte counts compared to controls. RBC counts in Hb S animals range from 2.5×10⁶ cells/μl to 3.6×10⁶ cells/μl; this is a 58-71% reduction from the number in control animals (8.5×10⁶ cells/μl). Similarly, Hb levels decrease from 15.0 g/dl in controls to 4.1-6.4 g/dl among sickle lines. PCVs decrease from a level of 45.7% in nonanemic control animals to levels ranging from 18.3 to 24.5% in Hb S mice. Because the spleen is a major hematopoietic tissue in anemic mice, Hb S animals attempt to compensate for severe hemolysis by increased erythropoiesis in their spleens. Individual spleens in these animals range from 7 to 20 times the mass of normal nonanemic mouse spleens. The spleens of some Hb S animals were 10% of the animal's body weight.

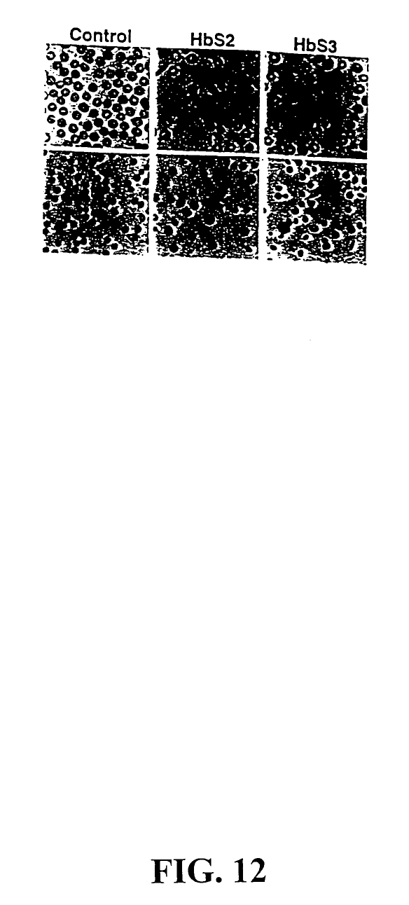

Peripheral blood smears of a control mouse and two Hb S lines at normal oxygen tension are shown in FIG. 12. Numerous sickled cells are observed in the blood of all the Hb S lines as exhibited in the Nb S2 and Hb S3 samples. Similar to peripheral blood smears of human Hb SS patients, anisocytosis and poikilocytosis are evident, and target cells and nucleated RBCs are observed. In contrast to man, sickle cell mice have a marked polychromasia indicative of large numbers of reticulocytes in the peripheral blood commensurate with increased erythropoiesis. This reticulocytosis results from severe anemia and is consistent with the marked expansion of erythropoiesis observed in the spleen of these mice. Reticulocyte counts in the Nb S mouse lines average 51 to 68%, compared to 3.9% in controls (FIG. 12, bottom panels); however, there is significant variability between individuals of the same line (Table 1). The hemolytic anemia described above develops during the first few weeks of life as the level of Hb F declines in these mice; this temporal pattern of onset mimics the onset of anemia in human sickle cell infants during the first few months of life.

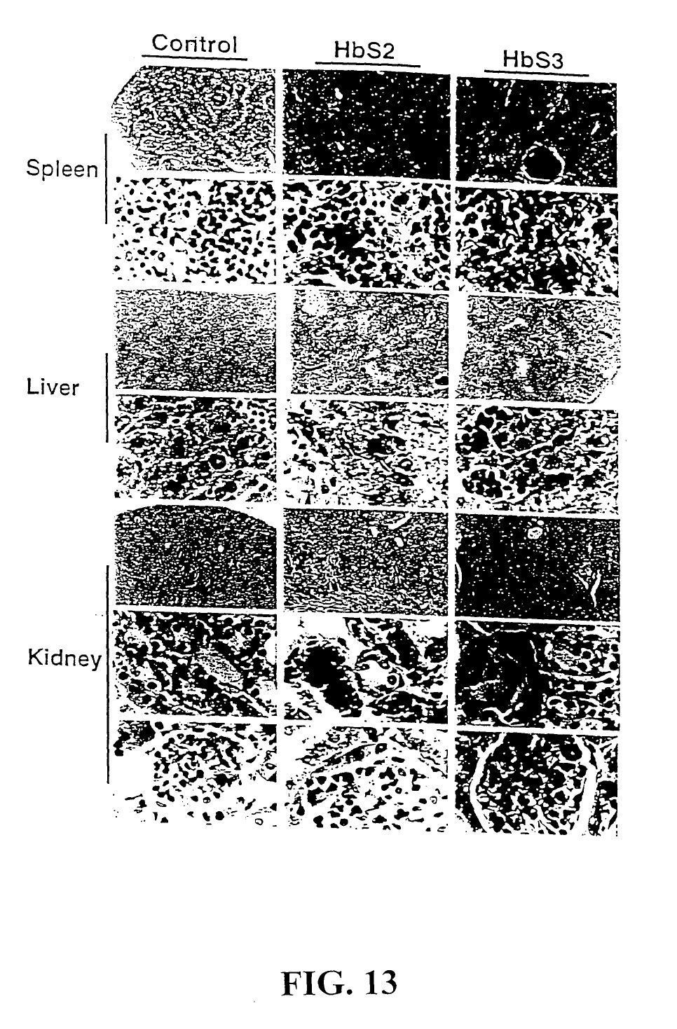

Unlike earlier mouse models of sickle cell disease, knock out/transgenic Nb S mice develop significant in vivo pathology at a relatively young age under ambient conditions. Spleen, liver, and kidney sections from seven week old control, Hb S2, and Hb S3 mice are shown in FIG. 13. Similar pathology was observed in all of the Hb S lines. Tissues were fixed in 70% alcoholic formalin, embedded in paraffin, sectioned, and stained with hematoxylin-eosin by standard methods. As mentioned previously, the spleens of sickle cell mice are massively enlarged. The normal splenic architecture of red and white pulp, as illustrated in the normal control, is obliterated in the Hb S mice by the large expansion of erythroid precursors. The red pulp (up to 95% of some spleens) is comprised of about equal numbers of pooled sickled RBCs and erythroid progenitors. Under higher magnification, dense mats of sickled erythrocytes are clearly observed in the splenic sinusoids. The extremely low RBC counts and Hb levels of sickle cell mice can be explained by the destruction of sickled erythrocytes in the peripheral circulation and by the large numbers of red blood cells that are continuously trapped inside the spleen. In humans, mean Hb levels of 4.8 g/dl have been reported in infants experiencing acute splenic sequestration (ASS) (Sedjeant, Brit J. Hemat. 19:635-641, 1970), and this value is similar to the levels found in Hb S mice (Table 1). Occasionally blood trapping leads to vascular occlusion and thrombosis. Many spleen sections contain an organizing thrombus similar to the one shown for Hb S3 in FIG. 13. Larger areas of mineralized tissue and amorphous acellular debris are also observed in older animals. This pathology is indicative of infarcts that occurred earlier in the life of the animals and implies that splenic function may decline over time. In humans, recurrent infarcts can result in autosplenectomy in childhood.

In the livers of Hb S mice, there is a generalized congestion of the intrahepatic vasculature and sinusoids with aggregates of sickled RBCs (FIG. 13). Extramedullary hematopoiesis occurs in the sinusoids as indicated by prominent clusters of erythroid precursors. Kupffer cell erythrophagocytosis with concomitant accumulation of hemosiderin is abundant. There is also periportal and subcapsular focal parenchymal necrosis. All of these pathologies are characteristic of the liver in human sickle cell patients (Bunn et al., Hemoglobin: Molecular, Genetic, and Clinical Aspects (W. B. Saunders, Philadelphia, 1986)).

In the kidneys of Hb S mice, engorgement and occlusion of blood vessels with sickled cells causes vascular, tubular, and glomerular changes (FIG. 13). Vascular occlusion is most prominent in the corticomedullary junction where engorged and dilated capillaries are easily observed. In man, reduced blood flow through the medullary region frequently causes extensive tubular damage resulting in hyposthenuria or the loss of urine concentrating ability. When Hb S3 mice (4-4.5 months old) are deprived of food and water for only 4 hours, the osmolality of the urine, as measured on a Wescor Inc. Model 5130C vapor pressure osmometer, was about half of control values (807+285 mOsm compared to 1541±360 mOsm). In younger animals, sickle cell congestion in the glomerulus and increased levels of iron deposition cause a mild membranoproliferative glomerulopathy. There is a moderate multifocal proximal tubular injury resulting in tubular dilatation, epithelial hypoplasia, basement membrane thickening, and iron deposition.

Despite the severe anemia and organ pathology in the Hb S mice, most animals survive for 2 to 9 months. Both male and female animals are fertile; however, litter sizes are much reduced (1-3 pups).

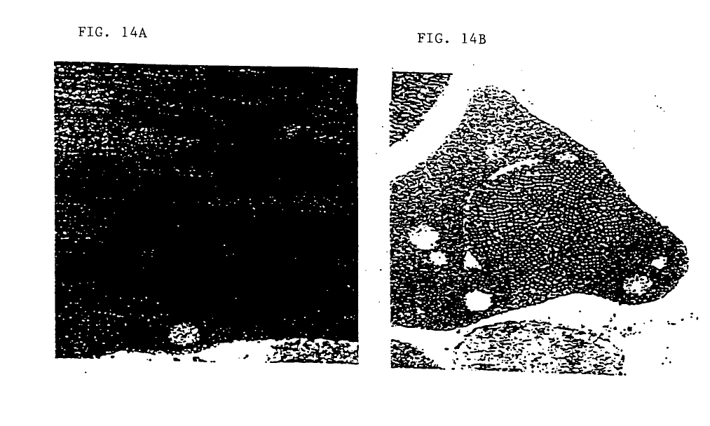

Finally, transmission electron micrographs (FIGS. 14A and 14B) of sickled erythrocytes in the sinusoids of an Hb S3 spleen reveals the presence of intracellular, human Hb S fibers under normal oxygen tensions. FIG. 14A illustrates the Hb S fibers in a RBC section in a plane parallel to the direction of the long axis of the fibers; FIG. 14B illustrates the ordered array of fibers in a transverse section. These fibers are indistinguishable from Hb S fibers present in erythrocytes of humans with sickle cell disease. The presence of these fibers reduces the flexibility of red blood cells and leads to the occlusion of small capillaries and subsequent organ damage.

The data described above demonstrate that mice containing human LCR γ-β^{S}/LCR α-globin transgenes combined with knock out mutations of the murine α- and β-globin genes synthesize 100% human hemoglobin in adult red blood cells. These novel, knock out/transgenic mice mimic many of the characteristics of sickle cell disease in humans. Similar to humans, the Hb S mice complete the switch from human Hb F to human Hb S after birth and subsequently develop a severe hemolytic anemia. Splenic infarction, liver necrosis, and loss of urine concentrating ability due to kidney tubular damage result in a chronic, deteriorating disorder that is characteristic of many patients with sickle cell disease. The animals of the invention are useful for testing novel drug and genetic therapies designed to treat and ultimately to cure sickle cell anemia.

The results described above were obtained using the following materials and methods. LCR α1 has been described previously (Ryan et al., Science 247:566-568, 1990). LCR ^{A}γ-β^{S} was constructed in a cosmid vector using DNase I hypersensitive sites 1-5 of the human β-globin locus control region (LCR) in a 22 kb SalI-ClaI fragment (Ryan et al., Genes & Dev. 3:314-323, 1989). The 5.65 kb ^{A}γ-globin gene fragment included in LCR ^{A}γ-β^{S} contains the ^{A}γ-globin gene from a HindIII site at −1348 to an EcoRI site at +4309. The 4.1 kb β^{S}-globin gene fragment included in LCR ^{A}γ-β^{S} contains the β-globin gene from a HpaI site at −815 to an XbaI site at +3285. LCR ^{A}γ-β^{S} fragments were excised from the cosmid vector and injected into C57B1/6×SJL fertilized mouse eggs. Transgenic founder animals were bred with C57B1/6×SJL mice to establish lines.

Genotyping of both the mouse ax- and β-globin loci was done by PCR on tail DNA in an MJ Research PTC100. PCR for the mouse a genotype used the following 3 oligonucleotides:

(1) mα1 FOR 5′-TCTTCTTGCCTCAGCCTACCAGG-3′ (SEQ ID NO:1) at nt −772 to −750 of the mouse (α1 promoter;

(2) mα1 REV 5′-CCCTCAAACCAAACTGAGGAGCG-3′ (SEQ ID NO:2) at nt −292 to −314; and

(3) neo REV 5′-TGAAGAGCTTGGCGGCGAATGGG-3′ (SEQ ID NO:3) at nt 687 to 709 with respect to the neomycin coding sequence; accession # V00618.

The wild type allele Hb A, designated mα⁺, is identified as a 480 bp product resulting from amplification with the mα1 FOR and mα1 REV primer combination. The knock out allele Hb A^{1-2del}, designated mα⁰, is identified by the mal FOR and neo REV primers giving rise to a 552 bp product.

Similarly, the mouse β locus genotype was determined using 3 oligonucleotides:

(1) mβ FOR 5′-GAGCAATGTGGACAGAGAAGGAG-3′ (SEQ ID NO:4) Genbank coordinates 37,875 to 37,897 at nt −412 to −390 of the mouse β1 promoter;

(2) my REV 5′-TGATGTCTGTTTCTGGGGTTGTG-3′ (SEQ ID NO:5) Genbank coordinates 38,339 to 38,317; accession # X14061; at nt 53 to 31 with respect to the mouse β1 transcriptional start site; and

(3) neo REV (see above).

The mβ FOR and mβ REV primer combination generates a 464 bp product indicative of the wild type Hbβ allele, designated mβ⁺. The mβ FOR and neo REV primers result in a 600 bp product diagnostic for the knock out Hbβ⁰ allele, designated mβ⁰. PCR conditions for amplifying the mouse α- and β-globin loci were identical. The reaction mixture minus polymerase was boiled for 5 minutes, cooled on ice, and enzyme was added. Amplification was accomplished by 30 cycles of PCR (92° C. for 20 seconds, 65° C. for 35 seconds, and 68° C. for 90 seconds) followed by one cycle of 10 minutes at 68° C.

Tail DNA was also screened by PCR for the transgenes. The LCR α1 transgene was identified by amplification of a 208 bp product using the following two oligonucleotides:

(1) LCR FOR 5′-AATATACCCTGACTCCTAGCCTG-3′ (SEQ ID NO:6) Genbank coordinates 18,615 to 18,637; accession # J00179; and

(2) hα1 REV 5′-CTGCAGGGTGAGGAAGGAAG-3′ (SEQ ID NO:7) Genbank coordinates 9,274 to 9,255; accession # J00153.

A 167 basepair product representing the LCR ^{A}γ-β^{S} was amplified using the LCR FOR primer and the hAγ REV primer 5′-ATGCCAGAAGCTCTGGAATTCTG-3′ (SEQ ID NO:8) Genbank coordinates 38,223 to 38,201; accession # J00179. For both LCRa and LCR Aγ-β^{S} PCR, amplifications were performed as described above except the annealing temperatures were 55° C. and 62° C., respectively.

Preparation of hemolysate samples for IEF analysis was carried out as follows. Blood from adult mice was collected in either EDTA Microtainer tubes (BD 365974) after cardiac puncture if the mouse was to be sacrificed; or into PBS, 2.5 mM EDTA if bled from the tail. Blood was washed three times with PBS before making hemolysates. Hemolysates were prepared and IEF was performed as described previously (Ryan et al., Science 247:566-568, 1990).

HPLC analysis of hemolysates was performed as follows. Reverse phase HPLC was performed on a Rainin Dynamax system using a Vydac C4 column (25×0.46 cm). Buffer A was 10% acetonitrile (ACN)/0.1% trifluoroacetic acid (TFA), and buffer B was 90% ACN/0.1% TFA. A non-linear gradient for one hour was used starting at 35% B to 36% B in the first minute, followed by an increase to 42% over the next 48 minutes, maintained at 42% B for 6 minutes, and returned to 35% B in the final 5 minutes. The flow rate was kept at 1 ml/minute and the effluent was monitored at 214 nm. Individual globin chains were quantitated with Dynamax HPLC Method Manager software.

The data in Table 1 was obtained using the following methods. Packed Cell Volumes (PCV) were determined by centrifugation of EDTA-treated blood in a Jorvet J503 centrifuge. Hemoglobin levels were determined at 540 nm by the cyanmethemoglobin method using a kit from Sigma (catalogue #525-A). Red blood cell counts were measured on a Coulter Counter (Model MHR).

The data shown in FIG. 12 were obtained as follows. Peripheral blood smears were made directly from a drop of tail blood under normal oxygen tension. Slides were stained with Wright-Giemsa stain. Reticulocytes were counted manually after supravital staining with 1% New Methylene Blue (Sigma catalogue # B4631) for 10 minutes at 37° C.

Samples for transmission electron microscopy were prepared as follows. A spleen from an Hb S mouse was fixed in one percent buffered glutaraldehyde and post fixed in one percent osmium tetroxide. After dehydration, the sample was embedded in polybed, sectioned, and stained with uranyl acetate and lead citrate. Ultrathin sections were examined and photographed with the Hitachi-7000 transmission electron microscope.

In addition to the method described above, mice expressing human sickle hemoglobin, but not mouse hemoglobin, can be generated using the methods described above in section I. For example, a targeting vector can be designed to simultaneously delete the mouse's adult α- and β-globin genes and replace them with the human α- and β^{S}-globin genes, respectively. The β^{s}-globin gene was introduced into the mouse β-globin locus by the scheme shown in FIG. 10. A correctly targeted ES cell clone was used to generate two chimeric males by the non-injection aggregation chimera technique. Progeny from a cross between these chimeric males and wild type females has demonstrated that the embryonic stem cell has contributed to the germline of both chimeras. Additionally, eight β^{s} chimeric mice have been produced by the blastocyst injection technique. We have shown that the transgene has been successfully passed through the germline.

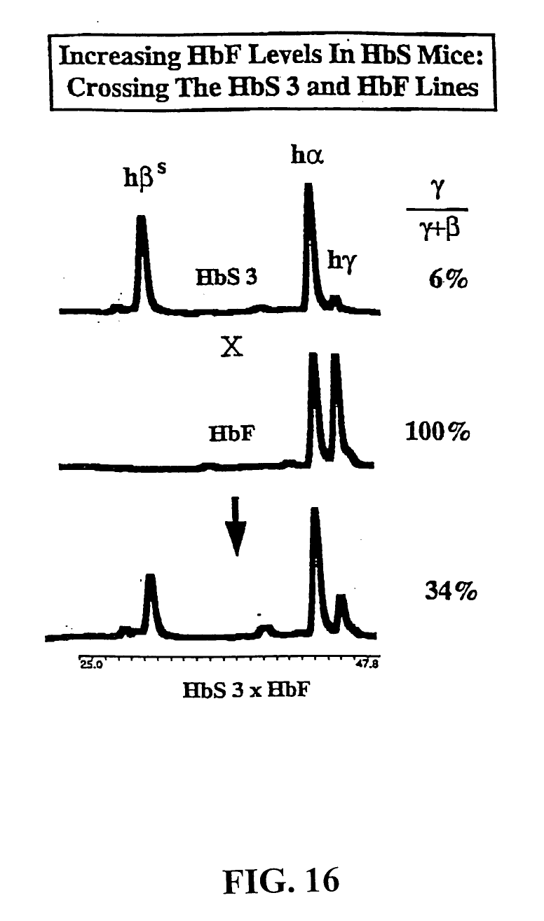

As is mentioned above, the transgenic, non-human mammals of the invention can be crossed with each other to generate additional animal models. For example, an Hb S mouse can be crossed with an Hb F mouse to study the effect of Hb F on sickling in Kb S mammals. An experiment illustrating this concept is shown in FIG. 16. Briefly, an Hb S3 mouse was crossed with an Hb F mouse. In the Hb S3 mouse, the γ/γ+β (%) was 6%, while in the Hb S mouse, the γ/γ+β (%) was 100%. In a mouse produced by crossing an Hb S3 mouse with an Kb F mouse, the γ/γ+β (%) was 34%.

IV. Crosslinked Hemoglobins

An additional type of hemoglobin that can be made in the transgenic animals of the invention is crosslinked hemoglobin, which is stabilized in tetrameric form when released from lysed red blood cells. Crosslinked hemoglobin can be useful as a red blood cell substitute. Hemoglobin tetramers rapidly dissociate into αβ dimers when red cells are lysed and the concentration of hemoglobin is decreased by dilution. To prevent tetramers from dissociating, internal, disulfide crosslinks can be introduced into human hemoglobin. These crosslinks stabilize α₂β₂ tetramers and, therefore, prolong the half-life of cell-free hemoglobin. The crystal structures of both deoxy- and oxy-hemoglobin have been accurately determined and the important sites of subunit interaction are known. The atomic distances were examined between various amino acids in areas of subunit interaction and several sites were identified in which cysteine substitutions for the normal amino acids in α and β polypeptides result in the formation of disulfide bridges between these chains. Those sites that would allow bond angles that favor disulfide linkage were chosen for mutagenesis.

Stabilization of human hemoglobin tetramers requires disulfide crosslinks between the two αβ dimers. Crosslinks could be between the α1 and β2 subunits, the a1 and a2 subunits, or the β1 and β2 subunits. Computer-assisted modeling and energy minimization were utilized to identify the sites in which cysteine substitutions for the normal amino acids would lead to the most stable disulfide bridges. The most stable tetramer disulfide bridges thus determined include bridges from:

1) a1 92 to β2 40,

2) β1 1 to β2 146, and

3) α2 130 to a cysteine added to the carboxyl terminus of the a 1 chain, designated α1 142.

Of the above disulfide bridges, a preferred one is an α1 92 to β2 40 (or α2 92 to β1 40) crosslink because a disulfide in this position would not hinder the rotations of cop dimers with respect to each other during the cooperative binding of oxygen.

Intermolecular Disulfides for Polymerization of Hemoglobin Tetramers

In addition to stabilizing the tetramer, disulfide bridges can also be used to link tetramers together to form polymers, such as octomers and the like. Tetramers stabilized by chemical crosslinking have a half-life of only 4 hours in vivo. Although the tetramers have a molecular weight of 64,000, they are filtered by the kidneys and can cause renal damage. Linkage of 2 tetramers produces a molecule of about 128,000 daltons. It has been demonstrated that octomers and higher molecular weight polymers produced by chemical crosslinking have a half-life of 40-48 hours in vivo and these molecules are not filtered by the kidneys (Gould et al., Ann. Surg. 211:394-398, 1990).

Another important advantage of polymerization of hemoglobin relates to the osmotic property of the polymer. The highest concentration of a crosslinked tetramer that would be iso-osmotic is 7 g/dl. However, this concentration does not provide sufficient oxygen carrying capacity (Gould et al., supra). An octomeric polymer would be iso-osmotic at 14 g/dI, which is the physiologic hemoglobin concentration. Hence, the crystal structures of deoxy- and oxy-hemoglobin were examined to determine the best position for a disulfide bridge between 2 tetramers. It was found that changing the a 1 aspartic acid 75 to cysteine produces a molecule capable of forming intermolecular crosslinks. Once an octomer is formed, steric hindrances inhibit further polymerization.

Alternative Self-Limiting Polymerization Strategy

As an alternative to the polymerization strategy described above, a naturally-occurring mutation that also results in polymerization was examined. This mutation is known as Hemoglobin Porto Alegre and involves a change from serine to cysteine at position 9 of the beta chain (Tonda et al., Amer. J. Human Genetics 15:265-279, 1963; Bonaventura et al., Science 158:800-802, 1967).

Hemoglobin (Hb) Porto Alegre polymerizes in a self-limiting fashion to form octomers composed of two hemoglobin tetramers or dodecomers composed of three tetramers (Bonaventura et al., supra; Tonda, An. Acad. Brasil. Cienc. 43:651-669, 1971). Although this hemoglobin does not polymerize in vivo, it forms stable polymers in vitro after exposure to gentle oxidizing conditions. After polymerization in vitro, polymers of Hb Porto Alegre are stable in reducing conditions similar to those present in serum (Tonda et al., An. Acad. Brasil. Cienc. 57:497-506, 1985). Therefore, it was postulated that genetically modified polymers would be ideally suited to function as a blood substitute. One undesirable characteristic of Hb Porto Alegre, however, is its increased oxygen affinity. In order to overcome this limitation, a second, oxygen affinity decreasing mutation can be made, as described below.

Approximation of Normal Oxygen Affinity in Hemoglobin Porto Alegre

The oxygen affinity of human hemoglobin is regulated by the molecule 2,3-diphosphoglycerate (DPG). Outside of red blood cells, DPG diffuses away from hemoglobin, resulting in a large increase in the hemoglobin's oxygen affinity. The resulting loss of DPG regulation can be compensated for by modification of the human hemoglobin so that its oxygen affinity approximates that of bovine hemoglobin.

Bovine hemoglobin has a naturally low oxygen affinity, which is not dependent upon DPG. Perutz et al. (J. Mol. Biol. 136:183-191, 1980) characterized the amino acid change responsible for the decreased oxygen affinity of bovine hemoglobin. The change occurs at the amino terminus of the beta chain and involves replacement of a hydrophilic residue at position NA2 with a hydrophobic residue. The modification of human hemoglobin to approximate that of bovine hemoglobin involves the removal of the first two amino acids at the N-terminus of the beta chain and their replacement by the hydrophobic amino acid methionine. The resulting β-globin polypeptide is composed of 145 amino acids instead of 146 and mimics the bovine β-globin chain at the amino terminal end.

As mentioned above, a second mutation, designed to counteract the increase in oxygen affinity of Hb Porto Alegre, can also be used. One such mutation, which occurs naturally, is known as Hb Kansas. In Kb Kansas, the beta 102 asparagine is changed to threonine (Bonaventura et al., J. Biol. Chem. 243:980-991, 1968). This mutation stabilizes the T, or Tense, conformation of hemoglobin, which is the structure normally found in venous blood after oxygen has been delivered to the tissues. The oxygen affinity of Hb Kansas is 2 fold lower than that of normal Hb A. Thus, it was postulated that Hb Kansas may decrease the abnormally high affinity associated with Hb Porto Alegre. Combinations of Hb Porto Alegre and Hb Kansas, as well as Hb Porto Alegre and the bovine mutations, were constructed. These combinations of mutant hemoglobins can be used as blood substitutes.

Other Genetic Modifications of Human Hemoglobins Synthesized in Transgenic Animals

Additional genetic modifications of human hemoglobin can be used in the invention. Computer-assisted modeling and energy minimization were employed to identify the sites in which cysteine substitutions for the naturally-occurring amino acids would lead to the most stable disulfide bridges. Of course, following this strategy, any number of new designs of these hemoglobin molecules can be generated. The basic strategy for identifying sites for cysteine substitution is as follows. The molecular coordinates of hemoglobin obtained from the Brookhaven Data Bank were loaded into an Evans and Sutherland PS300 Computer Graphics System. Cysteine substitutions were made at a variety of positions. Bond angles between pairs of cysteine residues on α1 and β2 chains were adjusted such that β carbon atoms were separated by less than 3.5 angstroms and disulfide bonds were formed between these residues. The disulfide linked tetramer was then subjected to energy minimization as described by Powell (Mathematical Programing 12:241-254, 1977) on a silicon graphics IRIS-4D. Briefly, energy minimization was conducted using the Powell method conjugate gradient minimizer provided in the software system X-PLOR version 2.1 (Brunger, X-PLOR: A System for Crystallography and NMR, Yale University, New Haven, 1990). Twenty five hundred cycles of minimization were conducted using both the oxy- and deoxyhemoglobin molecular coordinates. This established a baseline minimal total energy to which hemoglobins with engineered disulfides could be compared. The engineered hemoglobin with a disulfide bond from α1 92 to β2 40 displayed energy minima that were similar to those of the native human hemoglobin in both the deoxy- and oxygenated conformations. This bridge was subsequently selected as the first disulfide to be engineered for tetramer stabilization by site-directed mutagenesis. Specific cysteine codons were then introduced into α and β-globin genes by site-specific mutagenesis. Additional modifications to be incorporated into the design of mutant hemoglobins for use as a blood substitute, including combinations of naturally occurring mutants and those specifically designed by computer modeling, can be used in the invention.

Mutagenesis of Human α- and β-Globin Genes

Mutations were introduced into the normal human α- and β-globin genes by site-directed mutagenesis. A 3.8 kb BglII-EcoRI fragment containing the human (X-globin gene and a 4.1 kb HpaI-XbaI fragment containing the human β-globin gene were cloned into the pSELECT plasmid (Lewis et al., Nucl. Acids Res. 18:3439-3443, 1990) by standard procedures (Maniatis et al., Molecular Cloning: A Laboratory Manual, Cold Spring Harbor Laboratory Press, Cold Spring Harbor, N.Y., 1989). Oligonucleotide mutagenesis was performed as described by Lewis et al. (Nucl. Acids Res. 18:3439-3443, 1990). In this procedure an oligonucleotide that corrects a mutation in the ampicillin resistance gene in the pSELECT plasmid is used simultaneously with one or more oligonucleotides designed to create mutations in the globin gene insert. Briefly, E. coli (JM109) containing the pSELECT plasmid with globin gene inserts were infected with helper phage (M13K07). After growing the culture overnight (about 12-16 hours), phage obtained from the supernatant were extracted with phenol:chloroform and single-stranded DNA was isolated by standard methodology. Oligonucleotides containing each of the mutations were annealed to single-stranded DNA together with the wild type ampicillin oligonucleotide and these primers were extended with Klenow for about 90 minutes at 37° C. Double-stranded DNA was transformed into E. coli (BMH 71-18 mutS) and the culture was grown overnight in L-broth containing 75 μg/ml ampicillin. DNA obtained from rapid lysis preparations of these cultures was transfected into E. coli (JM109) and colonies were selected on ampicillin plates (75 μg/ml). Double-stranded DNA obtained from rapid lysis preparations of these colonies was sequenced (Sanger et al., Proc. Natl. Acad. Sci. USA 74:5463-5467, 1977) with oligonucleotides located upstream of the mutagenic oligonucleotides. Mutants were clearly identified by comparison to the wild type sequence. The oligonucleotides used to generate the mutations include those listed below. Underlined bases indicate the bases that differ from the wild type.

(1). Tetramer intramolecular crosslinks

A. α92 arginine to cysteine

CGG to TGC

5′-GCGCACAAGCTTTGCGTGGACCCGGTC-3′ (SEQ ID NO:9)

B. β40 arginine to cysteine

AGG to TGT

5′-CCTTGGACCCAGTGTTTCTTTGAGTCC-3′ (SEQ ID NO:10)

(2). Polymerization intermolecular crosslinks

A. α75 aspartic acid to cysteine (α octomer)

GAC to TGC

5′-CGCACGTGGACTGCATGCCCAACGC-3′ (SEQ ID NO:11)

B. β9 senrne to cysteine (Porto Alegre)

TCT to TGT

5′-CCTGAGGAGAAGTGTGCCGTTACTGCC-3′ (SEQ ID NO:12)

(3). Mutations to lower oxygen affinity

A. β102 asparagine to threonine (Hb Kansas)

AAC to ACC

5′-GTGGATCCTGAGACCTTCAGGGTGAGT-3′ (SEQ ID NO:13)

B. Bovine mutation (βΔ1-2) in which the first and second codons, GTG (valine) and CAC (histidine), are deleted

5′-CAAACAGACACCATGCTGACTCCTGAG-3′ (SEQ ID NO:14)

The wild type DNA sequence is ATGGTGCACCTGACT (SEQ ID NO:15) and the mutated sequence is ATG CTG ACT. The wild type amino acid sequence is Met-Val-His-Leu-etc. (SEQ ID NO:16). The methionine is cleaved from the amino terminal end by an aminopeptidase and the final protein is composed of 146 amino acids. The amino acid sequence of the mutant is Met-Leu-etc. The methionine is not removed from the amino terminal end because the aminopeptidase does not cleave the Met-Leu peptide bond. The final protein is thus composed of 145 amino acids.

The α75 and α92 mutations were introduced simultaneously into the α-globin gene with two separate oligonucleotides. The β40 and Bovine (βΔ1-2) mutations were introduced into the β-globin gene in a single mutagenesis with two different β-globin oligonucleotides. Similarly, the β40 and Kansas mutations were also introduced in the β-globin gene in a single mutagenesis with two different β-globin oligonucleotides. The Porto Alegre (β9) and Hb Kansas (β102) mutations were also introduced into the β-globin gene in a single mutagenesis with two different β-globin oligonucleotides. The Porto Alegre and bovine (βΔ1-2) mutations were created with a single 48 base oligonucleotide.

Construction of Cosmid Clones

Mutant α- and β-globin genes were excised from pSELECT plasmids and subcloned into “right arm” plasmids containing a Cos site. Specifically, a 1.2 kb NcoI-XbaI fragment from the α-globin pSELECT plasmids and a 1.4 kb ClaI-BamHI fragment from the β-globin pSELECT plasmids were inserted into right arm plasmids in place of the corresponding α- and β-globin gene wild type fragments. The α-globin right arm plasmids were digested with ClaI and MluI and 4.8 kb fragments containing mutated α-globin genes that were linked to Cos sites were purified from agarose gels. The β-globin right arm plasmids were digested with ClaI and HindIII and 6.5 kb fragments containing mutated β-globin genes that were linked to Cos sites were purified from agarose gels. Cosmids containing these fragments were constructed in four way ligations (Ryan et al., Genes. Dev. 3:314-323, 1989). The left arms were 9.0 kb MluI-SalI fragments obtained from the cosmid vector pCV001 (Lau et al., Proc. Natl. Acad. Sci. USA 80:5225-5229, 1983). This fragment contained a Cos site, an ampicillin resistance gene, a ColE1 origin, and the SVneo gene. The two internal fragments were a 10.7 kb SalI-KpnI fragment containing DNase I super-hypersensitive sites (HS) V, IV, and III and a 10.9 kb KpnI-ClaI fragment containing HS II and I. The four fragments were ligated together in a 2:1:1:2 molar ratio of vector arms to inserts and packaged (Packagene; Promega). E. coli ED8767 was infected with the packaged cosmids and plated onto ampicillin plates. Large scale cultures of ampicillin-resistant colonies were grown and cosmids were prepared by standard procedures.

Production of Transgenic Animals

Cosmid DNA was prepared by standard procedures. HS I-V α and HS I-V β cosmids containing the mutations described above were injected directly into fertilized mouse eggs or the constructs were digested with SalI and insert DNA was separated from plasmid DNA by agarose gel electrophoresis before injection. The eggs were injected and transferred to pseudopregnant foster mothers (Brinster et al., Proc. Natl. Acad. Sci. USA 82:4438-4442, 1985) and transgenic progeny were identified by Southern blot hybridization of tail DNA. Similarly, large animal eggs can be injected with the same constructs and transferred to foster mothers as described by Pursel et al. (Science 244:1281-1288, 1989).

Typically, human α- and β-globin genes were cloned into expression vectors designed to direct high levels of α- and β-globin synthesis in erythroid cells of transgenic animals. These constructs were coinjected into fertilized mouse eggs and expression was analyzed in transgenic animals that developed. All of the mice that contained intact copies of the transgenes expressed correctly initiated human α- and β-globin mRNA specifically in erythroid tissue. Isoelectric focusing of hemolysates demonstrated that a complete human hemoglobin was formed in adult erythrocytes and oxygen equilibrium curves of human hemoglobin purified from these mice demonstrated that the molecule was fully functional. The animals are healthy and faithfully transmit the human genes to progeny. These animals have been bred for over 20 generations and the progeny continue to synthesize equal amounts of human and mouse hemoglobins. It is pointed out that similar methodology can be used to produce functional (capable of efficiently delivering oxygen to tissues) human hemoglobin in large animals, such as pigs, sheep, goats, cows and the like.

Analysis of Blood from Transgenic Animals

Blood collected from transgenic animals is washed with saline and hemolysates prepared as described by Ryan et al. (Science 245:971-973, 1990). Hemoglobin is analyzed on isoelectric focusing (IEF) gels (Ryan et al., 1990, supra). Human hemoglobin bands are excised from IEF gels and analyzed on urea cellulose acetate strips to demonstrate that the human hemoglobin band is composed of human α- and β-globin polypeptides. It is noted that if human hemoglobin is difficult to separate from endogenous hemoglobins, mutations that increase or decrease the isoelectric point (pI) of human hemoglobin can be introduced into the α- and β-globin genes. Increases in pI are accomplished by introducing basic (positively charged) amino acids into the protein and decreases are accomplished by introducing acidic (negatively charged) amino acids. These charged amino acids are introduced at positions that do not disturb the structure or function of the protein. Oxygen equilibrium curves of purified hemoglobin are then determined as described by Ryan et al. (1990, supra).

Formation of Disulfide Crosslinks

Disulfide crosslinks in proteins are not easily formed inside erythrocytes because high concentrations of glutathione prevent oxidation (Tonda et al., 1985, supra). Both intramolecular and intermolecular disulfide crosslinks are formed after human hemoglobin is purified by isoelectric focusing as described above. Large scale purifications are accomplished by chromatofocusing (Giri, Methods. Enzymol., 182:380-392, 1990), which also separates proteins according to their isoelectric points. Purified human hemoglobin is then incubated for several days at 4° C. in slightly alkaline conditions (0.1 M Tris-HCL pH 8.0; Matsumura et al., Proc. Natl. Acad. Sci. USA 86:6562-6566, 1989) to gently oxidize the protein without oxidizing heme groups. Crosslinked hemoglobins are dialyzed into phosphate-buffered saline at pH 7.5 by tangential flow ultrafiltration (Shiloash et al., Adv. Biotechnol. Processes 8:97-125, 1988) against membranes that retain polymers of greater than 100,000 MW. These purified proteins are then analyzed on reducing and non-reducing polyacrylamide gels. Also, the oxygen equilibrium curves of these samples are obtained. Finally, the hemoglobins are tested for oxygen carrying capacity in animals following standard procedures.

It is noted that since the transgenically produced human hemoglobin is isolated in substantially pure form, free of any cellular or subcellular component, it is non-immunogenic; hence, useful as a blood-substitute without the need for blood typing, which becomes necessary if whole blood or red blood cells (RBCs) are to be used. In addition, being of animal origin, the transgenic hemoglobin is free of such viruses as HIV.

A composition in accordance with the present invention contains a biologically functional amount (i.e., capable of effective oxygen exchange with the tissues) or a blood substituting amount of the substantially pure transgenic human hemoglobin and a pharmaceutically acceptable vehicle such as physiological saline, non-toxic, sterile buffered medium, human plasma, and the like.

The availability of the substantially pure, cell-free, non-immunogenic, biologically functional, non-toxic, polymeric, transgenic human hemoglobin provides a method for supplementing the oxygen exchange capacity of the red blood cells (RBCs) by substituting the RBCs or the naturally occurring (wild type) whole blood with transgenic hemoglobin. The recombinant hemoglobin is particularly suitable, at least as a temporary substitute, for providing oxygen to tissues during critical times, such as during emergency surgery or until whole blood transfusions can be given, or for entirely obviating the need for whole blood transfusions. Of course, it can also be employed for organ perfusion and the like.

Production of Transgenic Kansas Porto Alegre Hemoglobin

Kansas and Porto Alegre mutations were introduced into the beta chain of human hemoglobin and expression constructs were produced as described above. These expression constructs were introduced into mice (also as described above) to produce transgenic animals expressing this mutant hemoglobin. Each chain of the human and mouse hemoglobins is expressed and is stable in vivo. Standard hemoglobin isolation from the animals, followed by anion exchange high performance liquid chromatography under denaturing conditions, was carried out. HPLC chromatogram analysis demonstrates that 34% of the total β-globin content of the animals is human Kansas Porto Alegre β-globin, and 41% of the total α-globin is human α-globin. Oxygen affinity curves, generated by standard techniques, were prepared using blood from the Kansas Porto Alegre mice (i.e., 40% human/60% mouse hemoglobin). The total hemoglobin in Kansas Porto Alegre transgenic mice exhibits a β50 of 23.5 mm Hg. This value can be compared to normal mouse hemoglobin (13 mm Hg), Porto Alegre hemoglobin (6 mm Hg), Kansas hemoglobin (36 mm Hg), and normal human hemoglobin (10 mm Hg) under similar conditions (0.1 M phosphate, pH 7.0; 20° C.). Under physiological conditions, the presence of 2,3-diphosphoglycerate (DPG) in intact red blood cells raises the β50 of normal human hemoglobin to 25. The cell-free hemoglobin used for a blood substitute will function in the low DPG environment of serum. Therefore, the P₅₀ of genetically modified hemoglobin should be in the range of 25-35. Both the P₅₀ of the transgenic Kansas Porto Alegre hemoglobin and the fact that viable transgenic offspring are produced indicates that this mutant hemoglobin properly binds oxygen and delivers it to tissues, and thus is functional in vivo.

V. Anti-Sickling Hemoglobins

Additional hemoglobins that can be produced in the transgenic animals of the invention are anti-sickling hemoglobins. As is discussed above, the molecular basis for sickle cell disease is an A to T transversion in the 6^{th} codon of the human β-globin gene. This simple transversion changes a polar glutamic acid residue to a non-polar valine (Ingram et al., Nature 178:792, 1956; Ingram et al., Nature 180:326, 1957) in the β-globin polypeptide and, thus, drastically decreases the solubility of this hemoglobin (termed Hb S).

Anti-Sickling β-globin Genes Designed to Inhibit Hb S Polymerization

Recombinant hemoglobins that contain anti-sickling mutations can be used to inhibit Hb S polymerization, and thus facilitate therapies for sickle cell anemia. In particular, the glutamic acid to valine change at the 6^{th} position of the β^{s} polypeptide creates a non-polar surface that readily interacts with a natural hydrophobic pocket in the β chain of a second tetramer. This natural pocket is formed primarily by a phenylalanine (phe) at position 85 and a leucine (leu) at position 88. This interaction leads to the formation of complex 14-stranded fibers (Bunn et al., Hemoglobin: Molecular, Genetic, and Clinical Aspects (W. B. Saunders, Philadelphia, 1986).

The structure of the fiber that forms in sickle erythrocytes was derived from X-ray diffraction studies of Hb S crystals (Edelstein, J. Mol. Biol. 150:557, 1981). Hb S tetramers are composed of two α-globin subunits (α₂) and two β^{S}-globin subunits (β^{s}₂), and form characteristic double stranded fibers. Interactions along the long axis of the fiber are termed axial contacts, while interactions along the sides of tetramers are lateral contacts (Bunn et al., supra). The β6 valine plays a critical role in the lateral contact by interacting with the hydrophobic residues β85 phenylalanine and β88 leucine. Accordingly, to interfere with detrimental Hb S polymerization, this interaction and, thus, hydrophobic pocket formation should be disrupted. Because Hb A (α₂β₂) has these same hydrophobic residues and is readily incorporated into sickle fibers, it cannot be used for this purpose. Moreover, although disruption of this pocket represents the best approach for inhibiting Hb S polymerization, certain strategies have detrimental side effects. For example, although amino acid substitutions at β85 Phe and β88 Leu would interfere with pocket formation, these amino acids are also important for correct positioning of the heme moiety, and cannot be mutated without severely altering oxygen affinity (Dickerson et al., Hemoglobin. Structure, Function, Evolution, and Pathology, Benjamin/Cummings, Menlo Park, Calif., 1983).

A better approach for inhibiting Hb S polymerization is the use of a β87 threonine (Thr) to glutamine (Gln) substitution that disrupts the hydrophobic pocket, without inhibiting β-globin function (Perutz et al., Nature 219:902-909, 1968; Computer graphics generated using an Evans and Sutherland PS300 system running the package FRODO; Jones, Meth. Enz. 115:157, 1985). The long side chain of glutamine prevents the β6 Val from interacting with the hydrophobic pocket. Human γ- and δ-globin polypeptides both have such a glutamine at position 87, and both Hb F (α₂γ₂) and Hb A2 (α₂δ₂) have potent anti-sickling activity (Nagel et al., Proc. Natl. Acad. Sci., USA 76(2):670-672, 1979). Another naturally occurring human hemoglobin, designated Hb D Ibadan, also has anti-sickling activity (Watson-Williams et al., Nature 205:1273, 1965). This hemoglobin has a lysine at position 87 and its long side chain also projects across the hydrophobic pocket and inhibits interactions with the β6 Val.

Preferably, to produce a recombinant anti-sickling hemoglobin, the mutations described above, which interfere with a major lateral contact, are combined with a second mutation that interferes with an axial contact. One such axial contact-disrupting mutation is described as follows. The side chains of the amino acids lysine-17 (Lys), asparagine-19 (Asn), and glutamic acid-22 (Glu) project to form a surface that stabilizes the axial contact with another sickle hemoglobin tetramer (Dickerson et al., supra). Although mutations at residues 17 or 19 are detrimental, amino acid 22 can be mutated from glutamic acid to alanine (Ala) without an alteration in hemoglobin function (Bowman et al., Biochemical and Biophysical Research Communications 26(4):466-470, 1967; Bunn et al., supra). The negative charge of the glutamic acid side chain at this position plays a key role in stabilizing the axial contact because it interacts with the positively charged imidazole group of a histidine at position 20 in the a chain of the neighboring tetramer. The shorter nonpolar alanine side chain fails to stabilize this interaction, thus disrupting the axial contacts between sickle hemoglobin tetramers. Hb AS2 contains a glutamine at position 87, together with an alanine at position 22. Hb AS1 has the same 1322 alanine and asparagine at β80 is replaced by lysine. This 1380 lysine significantly inhibits sickling when present as a single site mutation in Hb A (Nagel et al., Nature 283:832-1980). The following 27-mer oligos were used for mutagenesis at the indicated amino acids in 1-globin: β22, 5′-GTGAACGTGGATGCCGTTGGTGGTGAG-3′ (SEQ ID NO:17); 180, 5′-GCTCACCTGGACAAGCTCAAGGGCACC-3′ (SEQ ID NO:18); and 1387, 5′-GGCACCTTTGCCCAGCTGAGTGAGCTG-3′ (SEQ ID NO:19).

Another anti-sickling mutation in the human 13-globin gene is the Hb G Szuhu mutant, which contains a β80 Asn to Lys mutation that has significant anti-sickling activity (Nagel et al., Proc. Natl. Acad. Sci. USA 76(2):670-672, 1979), but does not impair hemoglobin function (Kaufman et al., Human Heredity 25:60-68, 1975). This mutation is preferably combined with the β22 Glu to Ala mutation described above.

Alternatively, an α-globin mutation can be used to inhibit Hb S polymerization. One example of such an α-globin mutation is provided by the hemoglobin designated Hb Montgomery (Brimhall et al., Biochim. Biophys. Acta. 379(1):28-32, 1975), which contains an a48 leucine to arginine mutation. The 54 year old patient from which this mutation was isolated was homozygous for β^{s}, but had no history of painful sickle cell crises, jaundice, leg ulcers, or stroke, and was only mildly anemic (Prchal et al., Am. J. Med. 86(2):232-236, 1989).

Anti-sickling hemoglobin AS3 combines the mutations at β22 and β87, which are present in anti-sickling hemoglobin AS2, with an additional mutation that lowers the oxygen affinity of the recombinant hemoglobin. The goal is to produce an anti-sickling hemoglobin that delivers oxygen to tissues prior to sickle hemoglobin (Hb S). We have termed this concept “preferential deoxygenation.” If the anti-sickling hemoglobin delivers oxygen preferentially, Hb S will remain oxygenated and, therefore, will not polymerize.

The mutation that was selected to lower the oxygen affinity of the anti-sickling hemoglobin is a change from asparagine to lysine at position 108 of the β-globin chain. This is the mutation that is present in the naturally-occurring Hb Presbyterian (Moo-Penn et al., FEBS Letters 92:53-56, 1978). Hb AS3 has the following three mutations: (1) β22 glutamic acid to alanine, (2) β87 threonine to glutamine, and (3) β108 asparagine to lysine.

Two additional anti-sickling hemoglobins, AS4 and AS5, have been made and combine the mutations present in Hb AS2 at β22 and β87, with additional mutations that cause the β-globin subunit to become more negatively charged. In red blood cells, surface charge is a key determinant of the ability of α-globin and β-globin monomers to associate with each other to form dimers (Bunn, Blood 69:1-6, 1987). The alpha subunit is somewhat positively charged, while the beta subunit is somewhat negatively charged. By increasing the negative charge on the β-globin subunit, it is possible to increase its affinity for the α-globin subunit. Introduction of an additional negative charge in the anti-sickling hemoglobin provides PAs polypeptides with a competitive advantage for interacting with α-globin polypeptides. Consequently, α₂β^{AS}₂ tetramers form more efficiently than α₂β^{S}₂ tetramers.

Anti-sickling hemoglobins AS4 and AS5 combine the mutations present in AS2 with a mutation that increases the negative charge on the β-globin subunit. One mutation that increases the negative charge on the β-globin subunit but does not affect the normal functioning of the hemoglobin molecule is a change from lysine to glutamic acid at position 95. This mutation occurs naturally and is known as Hb N-Baltimore. The resulting change in charge is −2, since a positively charged lysine is replaced by a negatively charged glutamic acid. This change in charge also allows Hb AS4 and Hb S to be distinguished by isoelectric focusing. Hb AS4 has the following three mutations: (1) β22 glutamic acid to alanine, (2) 1387 threonine to glutamine, and (3) 1395 lysine to glutamic acid.

An additional mutation, which occurs naturally and is known to increase the ability of the β-globin subunit to compete for the α-globin subunit, is known as Hb J-Baltimore. This mutation consists of a change from glycine to aspartic acid at position 16 of the β-globin subunit. While this mutation adds only one additional negative charge to the β-globin chain (compared to the two negative charges added by the N-Baltimore mutation described above), the location of the negative charge is significant. In fact, Hb J-Baltimore competes even more effectively than Hb N-Baltimore for the α-globin subunit. Hb AS5 has the following three mutations: (1) β116 glycine to aspartic acid, (2) 122 glutamic acid to alanine, and (3) β87 threonine to glutamine.

Anti-sickling hemoglobins can contain any combinations of the individual mutations described above. For example, the β108, β95, and β16 mutations can occur alone, in combination with the β22 mutation, or in combination with the β22 mutation and either the β80 or either of the above-described β87 mutations.

Mutagenesis of Human α- and β-globin Genes

Mutations can be introduced into the normal human α- and β-globin genes by site-directed mutagenesis. For example, a 3.8 kb BglII-EcoRI fragment containing the human α-globin gene or a 4.1 kb HpaI-XbaI fragment containing the human β-globin gene can be cloned into the pSELECT plasmid (Lewis et al., Nucl. Acids. Res. 18:3439-3443, 1990; pSELECT is available from the American Type Culture Collection, Rockville, Md., ATCC# 68196) using standard methods (see, e.g., Maniatis et al., supra). Oligonucleotide mutagenesis is performed, e.g., as described by Lewis et al. (Nucl. Acids. Res. 18:3439-3443, 1990). In this procedure, an oligonucleotide that corrects a mutation in the ampicillin resistance gene in the pSELECT plasmid is used simultaneously with one or more oligonucleotides designed to create mutations in the globin gene insert.

Briefly, E. coli (JM109; ATCC# 53323) containing the pSELECT plasmid with globin gene inserts are infected with helper phage (M13K07). After growing the culture overnight (about 12-16 hours), phage obtained from the supernatant are extracted with phenol:chloroform, and single-stranded DNA is isolated by standard methods. Oligonucleotides containing each of the mutations are annealed to single-stranded DNA together with the wild type ampicillin oligonucleotide, and these primers are extended with Klenow for about 90 minutes at 37° C. Double-stranded DNA is transformed into E. coli (BMH 71-18 mutS), and the culture is grown overnight in L-broth containing 75 μg/ml ampicillin. DNA obtained from rapid lysis preparations of these cultures is transfected into E. coli (JM109), and colonies are selected on ampicillin plates (75 μg/ml). Double-stranded DNA obtained from rapid lysis preparations of these colonies is sequenced (Sanger et al., Proc. Natl. Acad. Sci. USA 74:5463-5467, 1977) using oligonucleotide primers located upstream of the mutagenic oligonucleotides. Mutants are identified by comparison to wild type sequence.

Construction of Cosmid Clones

Constructs used for microinjection are as described by Behringer et al. (Science 245:971, 1989), except that the gene for sickle hemoglobin is replaced with genes encoding anti-sickling hemoglobins. Mutations are introduced into the human β-globin gene by site-specific mutagenesis, as described above, and the mutant sequences are inserted downstream of a 22 kb DNA fragment containing the DNase I hypersensitive sites I-V (5′ HS I-V) of the β-globin LCR (Lewis et al., Nucleic Acids Res. 18:3439, 1990), as described in further detail below.

In order to construct cosmid clones containing mutant α- and β-globin genes, the mutant genes are excised from pSELECT plasmids and subcloned into “right arm” plasmids containing a Cos site. Specifically, a 1.2 kb NcoI-XbaI fragment from the α-globin pSELECT plasmids and a 1.4 kb ClaI-BamHI fragment from the β-globin pSELECT plasmids are inserted into right arm plasmids in place of the corresponding α- and β-globin gene wild type fragments. The α-globin right arm plasmids are digested with ClaI and MluI, and 4.8 kb fragments containing mutant α-globin genes, which are linked to Cos sites, are purified by agarose gel electrophoresis. The β-globin right arm plasmids are digested with ClaI and HindIII, and 6.5 kb fragments containing mutant β-globin genes, which are linked to Cos sites, are purified similarly. Cosmids containing these fragments are constructed in four way ligations (Ryan et al., Genes Dev. 3:314-323, 1989). The left arms are 9.0 kb MluI-SalI fragments obtained from the cosmid vector pCV001 (Lau et al., Proc. Natl. Acad. Sci. USA 80:5225-5229, 1983). This fragment contains a Cos site, an ampicillin resistance gene, a ColE1 origin, and the SVneo gene. The two internal fragments are a 10.7 kb SalI-KpnI fragment containing DNase I super-hypersensitive (HS) sites V, IV, and III, and a 10.9 kb KpnI-ClaI fragment containing HS II and I. The four fragments are ligated together in a 2:1:1:2 molar ratio of vector arms to inserts and packaged (Packagene; Promega, Madison, Wis.). E. coli ED8767 is infected with the packaged cosmids and is plated onto ampicillin plates. Large scale cultures of ampicillin resistant colonies are grown, and cosmids are prepared by standard procedures.

Other Embodiments

In addition to targeting and replacing (or knocking out) one gene at a time, both alleles of a gene or multiple genes can be targeted at once (Ausubel et al., supra). In addition, one skilled in the art can modulate gene dosage by inserting single or multiple copies of genes in the gene replacement methods described above.

From the above description, one skilled in the art can easily ascertain the essential characteristics of the invention and, without departing from the spirit and scope thereof, can make various changes and modifications of the invention to adapt it to various usages and conditions. All publications cited herein are fully incorporated by reference in their entirety. Other embodiments of the invention are in the claims set forth below.

What is claimed is:

1. A transgenic, non-human mammal comprising erythrocytes that produce a human hemoglobin, but fail to produce adult hemoglobin endogenous to said non-human mammal.

2. The transgenic, non-human mammal of claim 1, wherein said erythrocytes fail to produce non-adult hemoglobin endogenous to said non-human mammal.

3. The transgenic, non-human mammal of claim 1, wherein said transgenic, non-human mammal is a mouse.

4. The transgenic, non-human mammal of claim 1, wherein said human hemoglobin is hemoglobin A.

5. The transgenic, non-human mammal of claim 1, wherein said human hemoglobin is sickle hemoglobin.

6. The transgenic, non-human mammal of claim 1, wherein said human hemoglobin is fetal hemoglobin.

7. The transgenic, non-human mammal of claim 1, wherein said human hemoglobin is an anti-sickling hemoglobin.

8. The transgenic, non-human mammal of claim 7, wherein said anti-sickling hemoglobin is selected from the group consisting of Hb AS1, Hb AS2, Hb AS3, Hb AS4, and Hb AS5.

9. The transgenic, non-human mammal of claim 1, wherein said human hemoglobin is hemoglobin Kansas Porto Alegre.

10. The transgenic, non-human mammal of claim 1, wherein said erythrocytes produce human fetal hemoglobin and human sickle hemoglobin.

11. The transgenic, non-human mammal of claim 1, wherein precursors of said erythrocytes each comprise a human hemoglobin gene comprising a thalassemic mutation.

12. The transgenic, non-human mammal of claim 11, wherein said precursors of said erythrocytes each further comprise a gene encoding a human γ-globin chain.

13. The transgenic, non-human mammal of claim 11, wherein said precursors of said erythrocytes each further comprise a gene encoding a human β-globin chain.

14. The transgenic, non-human mammal of claim 1, wherein said erythrocytes produce a human anti-sickling hemoglobin and human sickle hemoglobin.

15. The transgenic, non-human mammal of claim 1, wherein precursors of said erythrocytes each comprise a chromosome comprising a human γ-globin gene and a human β-globin gene.

16. The transgenic, non-human mammal of claim 1, wherein precursors of said erythrocytes each comprise a first chromosome comprising a human γ-globin gene and a human β-globin gene, and a second chromosome comprising a human c-globin gene, a human γ-globin gene, a human 8-globin gene, and a human β-globin gene.

17. The transgenic non-human mammal of claim 16, wherein said human β-globin gene encodes a β^{s} hemoglobin chain.

18. The transgenic, non-human mammal of claim 16, wherein precursors of said erythrocytes each comprise a first chromosome comprising a human γ-globin gene and a human β-globin gene, and a second chromosome comprising a human ε-globin gene, two human γ-globin genes, a human ψβ-globin gene, a human δ-globin gene, and a human β-globin gene.

19. A method of producing human hemoglobin, said method comprising expressing said human hemoglobin in the erythrocytes of a transgenic, non-human mammal of claim 1.

20. Human hemoglobin produced by the method of claim 19.

21. A method of testing a substance for efficacy in treating sickle cell anemia, said method comprising exposing a transgenic, non-human mammal of claim 5 to said substance and monitoring a characteristic of sickle cell anemia in said transgenic, non-human mammal following substance exposure, wherein amelioration of said characteristic of sickle cell anemia indicates a substance useful for treating sickle cell anemia.

22. The method of claim 21, wherein precursors of said erythrocytes each comprise a first chromosome comprising a human γ-globin gene and a human β-globin gene, and a second chromosome comprising a human ε-globin gene, a human γ-globin gene, a human δ-globin gene, and a human β^{S}-globin gene.

23. The method of claim 21, wherein said characteristic of sickle cell anemia is red blood cell sickling.

24. The method of claim 21, wherein said transgenic, non-human mammal is a mouse.


SEQUENCE LISTING (19 sequences)

SEQ ID NO: 1 - DNA, 23 bp, Mus musculus
tcttcttgcctcagcctaccagg

SEQ ID NO: 2 - DNA, 23 bp, Mus musculus
ccctcaaaccaaactgaggagcg

SEQ ID NO: 3 - DNA, 23 bp, Escherichia coli
tgaagagcttggcggcgaatggg

SEQ ID NO: 4 - DNA, 23 bp, Mus musculus
gagcaatgtggacagagaaggag

SEQ ID NO: 5 - DNA, 23 bp, Mus musculus
tgatgtctgtttctggggttgtg

SEQ ID NO: 6 - DNA, 23 bp, Homo sapiens
aatataccctgactcctagcctg

SEQ ID NO: 7 - DNA, 20 bp, Homo sapiens
ctgcagggtgaggaaggaag

SEQ ID NO: 8 - DNA, 23 bp, Homo sapiens
atgccagaagctctggaattctg

SEQ ID NO: 9 - DNA, 27 bp, Homo sapiens
gcgcacaagctttgcgtggacccggtc

SEQ ID NO: 10 - DNA, 27 bp, Homo sapiens
ccttggacccagtgtttctttgagtcc

SEQ ID NO: 11 - DNA, 25 bp, Homo sapiens
cgcacgtggactgcatgcccaacgc

SEQ ID NO: 12 - DNA, 27 bp, Homo sapiens
cctgaggagaagtgtgccgttactgcc

SEQ ID NO: 13 - DNA, 27 bp, Homo sapiens
gtggatcctgagaccttcagggtgagt

SEQ ID NO: 14 - DNA, 27 bp, Homo sapiens
caaacagacaccatgctgactcctgag

SEQ ID NO: 15 - DNA, 15 bp, Homo sapiens
atggtgcacctgact

SEQ ID NO: 16 - protein, 4 aa, Homo sapiens
MVHL

SEQ ID NO: 17 - DNA, 26 bp, Homo sapiens
tgaacgtggatgccgttggtggtgag

SEQ ID NO: 18 - DNA, 27 bp, Homo sapiens
gctcacctggacaagctcaagggcacc

SEQ ID NO: 19 - DNA, 27 bp, Homo sapiens
ggcacctttgcccagctgagtgagctg